(19) United States
(12) Patent Application Publication
(10) Pub. No.: US 2025/0195490 A1
(43) Pub. Date: Jun. 19, 2025

(54) ALPHA-METHYLENE AND AMINOMETHYL LACTONES AND LACTAMS FOR TREATMENT OF CLOSTRIDIOIDES DIFFICILE INFECTION (CDI)

(71) Applicant: Purdue Research Foundation, West Lafayette, IN (US)

(72) Inventors: Padinjaremadhom V. Ramachandran, W. Lafayette, IN (US); Mohamed Seleem, Blacksburg, VA (US)

(73) Assignee: Purdue Research Foundation, West Lafayette, IN (US)

(21) Appl. No.: 18/294,814

(22) PCT Filed: Feb. 3, 2023
(86) PCT No.: PCT/US2023/061991

§ 371 Date: Feb. 2, 2024

Related U.S. Application Data
(60) Provisional application No. 63/306,164, filed on Feb. 3, 2022.

(51) Int. Cl. A61K 31/4525; A61P 31/04; C07D 307/33

(52) U.S. Cl. CPC A61K 31/4525; A61P 31/04; C07D 307/33

(57) ABSTRACT

Compounds to treat various infections, including infections caused by Clostridioides difficile, particularly the compounds α-methylene and α-aminomethyl lactones, lactams, iminolactones, and iminolactams, thiolactones, thionolactones, thiolactams, and thionolactams; pharmaceutical compositions; and methods for treating those infections.

### CROSS REFERENCE TO RELATED APPLICATIONS

This application claims priority to U.S. provisional patent application No. 63/306,164, which was filed on Feb. 3, 2022, which is hereby incorporated by reference in its entirety.

### TECHNICAL FIELD

This disclosure relates to compounds and methods for the treatment of a patient with a microbial, particularly to α-methylene and α-aminomethyl lactones, lactams, iminolactones, and iminolactams, thiolactones, thionolactones, thiolactams, and thionolactams for the treatment of infections caused by Clostridioides (previously referred to as Clostridium) difficile.

### BACKGROUND

This section introduces aspects that may help facilitate a better understanding of the disclosure. Accordingly, these statements are to be read in this light and are not to be understood as admissions about what is or is not prior art.

The discovery and development of antibiotics revolutionized health care in such a way that bacterial infections, which were otherwise deadly, could be treated. The most common side effects of the treatment of bacterial infection with antibiotics are diarrhea and colitis [1]. It is largely accepted that the perturbation of the normal intestinal microbiota caused by antibiotics provides suitable conditions for toxigenic bacterial growth, resulting in antibiotic-associated diarrhea (AAD). It has been almost four decades since toxigenic Clostridioides difficile (C. difficile) was identified as a common causative agent of AAD and colitis [2]. Today, roughly a third of AAD is attributable to C. difficile [3] with an estimated 500,000 cases annually resulting in ˜29,000 deaths in the United States alone [4].

Fidaxomicin is the only FDA-approved antibiotic during the past 40 years to combat Clostridioides difficile infection (CDI) since vancomycin [5-7]. However, recurrence rates for fidaxomicin are still high for infections involving the hypervirulent strains (24% recurrence rate) and for patients treated for an episode of recurrent C. difficile-associated diarrhea (20% recurrence rate) [8-11]. One out of every five patients with CDI experienced a recurrence of the infection, and one out of every nine patients aged 65+ died within 30 days of diagnosis [12]. Furthermore, the rates of death associated with CDI are rising, and the infection is occurring in populations considered to be at low risk previously, such as young and healthy adults [8]. Thus, there remains a desperate need to develop selective, new antibiotics with improved efficacy to treat CDI with little or no side effects.

In view of the foregoing, it is an object of the present disclosure to provide new drugs to treat CDI. This and other objects and advantages, as well as inventive features, will be apparent from the detailed description provided herein.

### SUMMARY

The disclosure relates to a compound having the formula

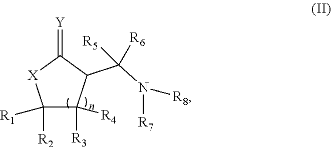

or a pharmaceutically acceptable salt thereof,
wherein
X is O, NH, NR, or S;
Y is O, NH, NR, or S;
n is 1, 2, 3, or 4;
R is H or alkyl;
R₁, R₂, R₃, R₄, R₅, and R₆ are independently hydrogen, halogen, hydroxyl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
R₇ and R₈ are independently hydrogen, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; or R₇ and R₈ are taken together with the N to which they are attached to form a ring system.

The disclosure also relates to a compound of formula (I), wherein the compound is of formula

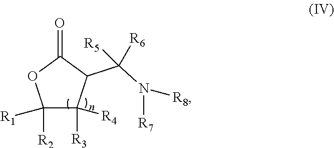

or a pharmaceutically acceptable salt thereof,
wherein
n is 1, 2, 3, or 4;
R₁, R₂, R₃, R₄, R₅, and R₆ are independently hydrogen, halogen, hydroxyl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
R₇ and R₈ are independently hydrogen, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; or R₇ and R₈ are taken together with the N to which they are attached to form a ring system.

The disclosure also relates to compound of formula

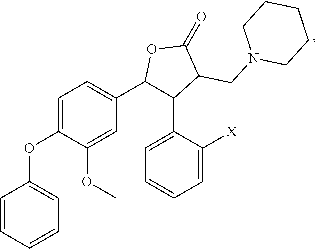

or a pharmaceutically acceptable salt thereof, wherein X is a halo.

The disclosure also relates to a compound of formula

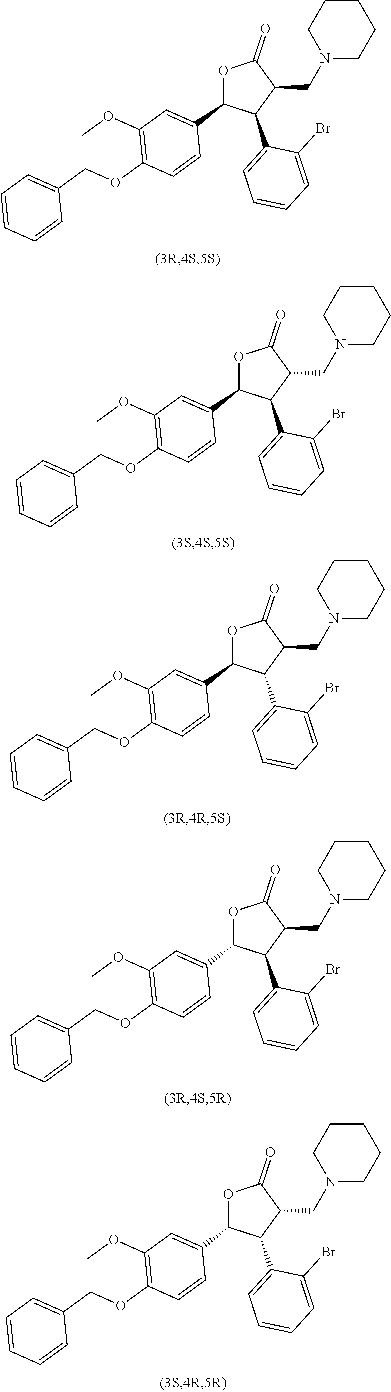 [SMILES: COc1cc([C@H]2OC(=O)[C@@H](CN3CCCCC3)[C@H]2c2ccccc2Br)ccc1OCc1ccccc1 ; COc1cc([C@H]2OC(=O)[C@H](CN3CCCCC3)[C@H]2c2ccccc2Br)ccc1OCc1ccccc1 ; COc1cc([C@H]2OC(=O)[C@@H](CN3CCCCC3)[C@@H]2c2ccccc2Br)ccc1OCc1ccccc1 ; COc1cc([C@@H]2OC(=O)[C@@H](CN3CCCCC3)[C@H]2c2ccccc2Br)ccc1OCc1ccccc1 ; COc1cc([C@@H]2OC(=O)[C@H](CN3CCCCC3)[C@@H]2c2ccccc2Br)ccc1OCc1ccccc1]

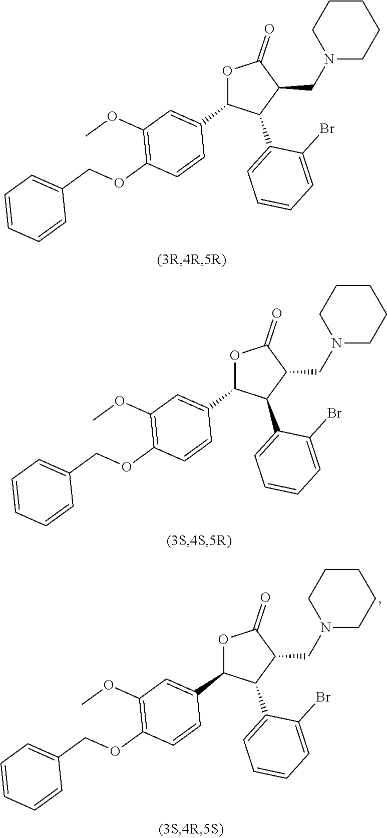 [SMILES: COc1cc([C@@H]2OC(=O)[C@@H](CN3CCCCC3)[C@@H]2c2ccccc2Br)ccc1OCc1ccccc1 ; COc1cc([C@@H]2OC(=O)[C@H](CN3CCCCC3)[C@H]2c2ccccc2Br)ccc1OCc1ccccc1 ; COc1cc([C@H]2OC(=O)[C@H](CN3CCCCC3)[C@@H]2c2ccccc2Br)ccc1OCc1ccccc1]

or a pharmaceutically acceptable salt thereof.

The disclosure also relates to a compound of formula (I):

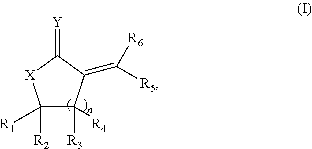

wherein
X is O, NH, NR, or S;
Y is O, NH, NR, or S;
n is 1, 2, 3, or 4; and
R₁, R₂, R₃, R₄, R₅, and R₆ are independently hydrogen, halogen, hydroxyl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted.

The disclosure also relates to a compound of formula (I), wherein the compound is of formula

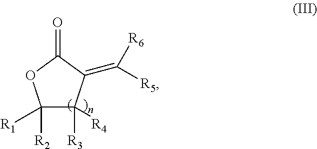

or a pharmaceutically acceptable salt thereof, wherein n is 1, 2, 3, or 4; and
R₁, R₂, R₃, R₄, R₅, and R₆ are independently hydrogen, halogen, hydroxyl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl.

The disclosure also relates to a pharmaceutical composition comprising one or more compounds of any of formula (I), (II), (III), or (IV), together with one or more pharmaceutically acceptable diluents, excipients or carriers. The pharmaceutical composition can be nanoparticulate. The pharmaceutical composition can comprise one or more other antibiotics.

The disclosure also relates to method for treating a patient with an infection comprising administering a therapeutically effective amount of one or more compounds of formula (I), (II), (III), or (IV), or a pharmaceutically acceptable salt thereof, or a pharmaceutical composition thereof to a patient in need thereof. The infection can be caused by Clostridioides difficile.

The disclosure also relates to a compound or a pharmaceutically acceptable salt thereof according to any of claims 1-6 in the manufacture of a medicament for treating Clostridioides difficile infection in a subject.

These and other features, aspects and advantages of the disclosure will become better understood with reference to the following detailed description and claims.

### BRIEF DESCRIPTION OF DRAWINGS

FIG. 1 shows the structures of lactones with selectivity against C. diff.

FIG. 2 shows toxicity analysis of compounds 27, 48, 50, 51, 61, and 85 against human colorectal cells (Caco-2). Percent viable mammalian cells (measured as average absorbance ratio (test agent relative to DMSO) for cytotoxicity analysis of the compounds (tested in triplicate) at 32, 64 and 128 μg/mL against Caco-2 cells using the MTS 3-(4,5-dimethylthiazol-2-yl)-5-(3-carboxymethoxyphenyl)-2-(4-sulfophenyl)-2H-tetrazolium) assay. Dimethyl sulfoxide (DMSO) was used as a negative control to determine a baseline measurement for the cytotoxic impact of each compound. The absorbance values represent an average of a minimum of three samples analyzed for each compound. Error bars represent standard deviation values for the absorbance values. A two-way ANOVA, with post hoc Dunnet's multiple comparisons test, determined statistical difference (denoted by the asterisk, *) (P<0.05) between the values obtained for each compound and DMSO (negative control, used as solvent for the compounds).

FIG. 3 shows mice sensitized to CDI with two different protocols A (antibiotic cocktail) and B (single antibiotic) and infected with C. difficile and treated with drugs.

FIG. 4 shows CDI mouse recurrence model.

FIG. 5A-5H shows results from histological toxicity examination of kidney (5A), liver (5B), lung (5C), spleen (5D), heart (5E), stomach (5F), small intestine (5G) and intestines (5H) tissues of mouse. Treated and control data are compared side by side. All images are 10× magnified.

### DETAILED DESCRIPTION

While the concepts of the present disclosure are illustrated and described in detail in the description herein, results in the description are to be considered as exemplary and not restrictive in character; it being understood that only the illustrative embodiments are shown and described and that all changes and modifications that come within the spirit of the disclosure are desired to be protected.

The disclosure generally relates to compounds useful for the treatment of infectious diseases. Pharmaceutical compositions and methods for treating those diseases are within the scope of this disclosure.

The disclosure relates to compounds useful for the treatment of an infectious disease. In embodiments, the disclosure relates to a compound having a formula

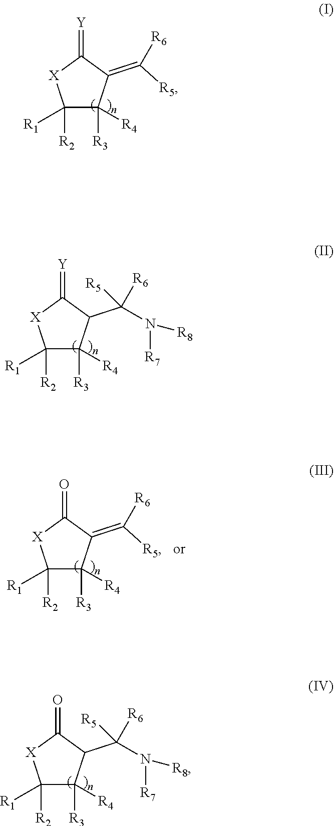

or a pharmaceutically acceptable salt thereof, wherein:
   X is O, NH, NR, or S;
   Y is O, NH, NR, or S;
   n is 1, 2, 3, or 4; and
   R₁, R₂, R₃, R₄, R₅, and R₆ in the general formulas are the same or different and can be independently a hydrogen atom, a halogen atom, a hydroxyl group, and any other linear, branched, or cyclic aliphatic group containing any number of carbon (1-30) atoms. These groups can contain one or more heteroatoms, such as oxygen, sulfur, or nitrogen. Representative groups include, but are not limited to, alkyl, substituted alkyl, alkenyl, substituted alkenyl, alkynyl, substituted alkynyl, aryl, substituted aryl (e.g., ortho-, and/or meta-, and/or para-substituted phenyl), heteroaryl, and substituted heteroaryl. Representative substitutions include, but are not limited to, alkoxy, alkylthio, halo, hydroxyl, phenoxy, aryloxy, cyano, isocyano, carbonyl, carboxyl, amino, amido, sulfonyl, substituted heterocyclic, or the like.
   R₇ and R₈ can be independently a hydrogen atom, linear, branched, or cyclic aliphatic groups, aryl groups, heteroaryl groups, or substituted aryl groups, or R₇ and R₈ are taken together to form a ring system with the N to which they are attached, such as in an aziridine, azetidine, pyrrolidine, or piperidine. These cycles can contain other heteroatoms, such as N, S, or O to form heterocycle such as morpholine or thiomorpholine.

In embodiments, the disclosure relates to a method or a process to prolong the clinical utility of current antibiotics, such as vancomycin and fidaxomicin.

In embodiments, the disclosure relates to a method of use of a compound, or a pharmaceutically acceptable salt thereof, in the manufacture of a medicament for treating infection in a subject.

In embodiments, the disclosure relates to a pharmaceutical composition comprising a compound, together with one or more pharmaceutically acceptable diluents, excipients or carriers.

In embodiments, the disclosure relates to a compound having the formula

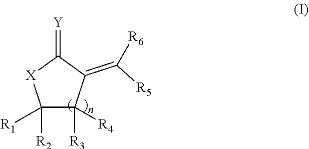

wherein:
   X is O, NH, NR, or S;
   Y is O, NH, NR, or S;
   n is 1, 2, 3, or 4;
   R is H or alkyl; and
   R₁, R₂, R₃, R₄, R₅, and R₆ are independently hydrogen, halogen, hydroxyl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted;
   or a pharmaceutically acceptable salt thereof.

In embodiments, the disclosure relates to a compound the formula

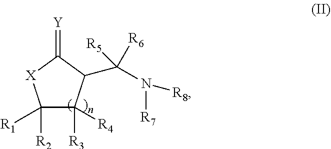

or a pharmaceutically acceptable salt thereof,
wherein
X is O, NH, NR, or S;
Y is O, NH, NR, or S;
n is 1, 2, 3, or 4;
R is H or alkyl;
R₁, R₂, R₃, R₄, R₅, and R₆ are independently hydrogen, halogen, hydroxyl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
R₇ and R₈ are independently hydrogen, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; or R₇ and R₈ are taken together to form a ring system with the N to which they are attached.

In embodiments, the disclosure relates to a compound for treating a patient with an infection having the formula

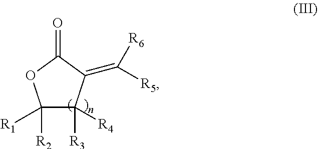

or a pharmaceutically acceptable salt thereof,
wherein
   n is 1, 2, 3, or 4; and
   R₁, R₂, R₃, R₄, R₅, and R₆ are independently hydrogen, halogen, hydroxyl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted.

In embodiments, the disclosure relates to a compound having the formula

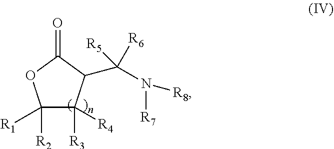

or a pharmaceutically acceptable salt thereof,
wherein
n is 1, 2, 3, or 4;
R₁, R₂, R₃, R₄, R₅, and R₆ are independently hydrogen, halogen, hydroxyl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
R₇ and R₈ are independently hydrogen, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; or R₇ and R₈ are taken together to form a ring system with the N to which they are attached.

In embodiments, the disclosure relates to a method for treating a patient with an infection comprising the step of administering a therapeutically effective amount of one or more compounds of formula (I), or a pharmaceutically acceptable salt thereof, together with one or more carriers, diluents, or excipients, to a patient in need of relief from said infection:

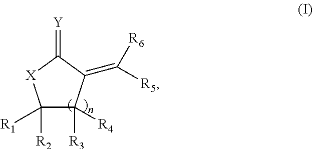

wherein
   X is O, NH, NR, or S;
   Y is O, NH, NR, or S;
   n is 1, 2, 3, or 4; and
   R₁, R₂, R₃, R₄, R₅, and R₆ are independently hydrogen, halogen, hydroxyl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted.

In embodiments, the disclosure relates to a method for treating a patient with an infection comprising the step of administering a therapeutically effective amount of one or more compounds of formula (I), or a pharmaceutically acceptable salt thereof, wherein said infection is caused by Clostridioides difficile.

In embodiments, the disclosure relates to a method for treating a patient with an infection comprising the step of administering a therapeutically effective amount of one or more compounds of formula (II), or a pharmaceutically acceptable salt thereof, together with one or more carriers, diluents, or excipients, to a patient in need of relief from said infection:

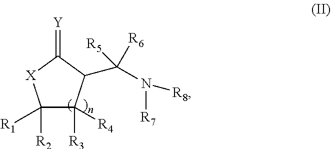

wherein
   X is O, NH, NR, or S;
   Y is O, NH, NR, or S;
   n is 1, 2, 3, or 4;
   R₁, R₂, R₃, R₄, R₅, and R₆ are independently hydrogen, halogen, hydroxyl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
   R₇-R₈ are independently hydrogen, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; or
   R₇ and R₈ are taken together to form a ring system with the N to which they are attached.

In embodiments, the disclosure relates to a method for treating a patient with an infection comprising the step of administering a therapeutically effective amount of one or more compounds of formula (II), or a pharmaceutically acceptable salt thereof, together with one or more carriers, diluents, or excipients, to a patient in need of relief from said infection, wherein said infection is caused by Clostridioides difficile.

In embodiments, the disclosure relates to a method for treating a patient with an infection comprising the step of administering a therapeutically effective amount of one or more compounds of formula (III), or a pharmaceutically acceptable salt thereof, together with one or more carriers, diluents, or excipients, to a patient in need of relief from said infection:

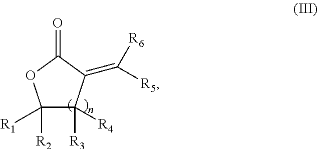

wherein
   n=1, 2, 3, or 4; and
   R₁, R₂, R₃, R₄, R₅, and R₆ are independently hydrogen, halogen, hydroxyl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted.

In embodiments, the disclosure relates to a method for treating a patient with an infection comprising the step of administering a therapeutically effective amount of one or more compounds of formula (III), or a pharmaceutically acceptable salt thereof, together with one or more carriers, diluents, or excipients, to a patient in need of relief from said infection, wherein said infection is caused by Clostridioides difficile.

In embodiments, the disclosure relates to a method for treating a patient with an infection comprising the step of administering a therapeutically effective amount of one or more compounds of formula (IV), or a pharmaceutically acceptable salt thereof, together with one or more carriers, diluents, or excipients, to a patient in need of relief from said infection:

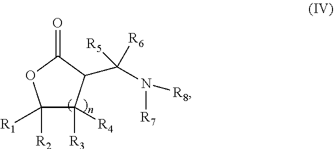

wherein:
   n is 1, 2, 3, or 4;
   R₁, R₂, R₃, R₄, R₅, and R₆ are independently hydrogen, halogen, hydroxyl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
   R₇ and R₈ are independently hydrogen, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; or R₇ and R₈ are taken together to form a ring system with the N to which they are attached.

In embodiments, the disclosure relates to a method for treating a patient with an infection comprising the step of administering a therapeutically effective amount of one or more compounds of formula (IV), or a pharmaceutically acceptable salt thereof, together with one or more carriers, diluents, or excipients, to a patient in need of relief from said infection, wherein said infection is caused by Clostridioides difficile.

In embodiments, the disclosure relates to the use of an above method to prolong the clinical utility of current antibiotics, such as vancomycin and fidaxomicin.

In embodiments, the disclosure relates to a compound having the formula

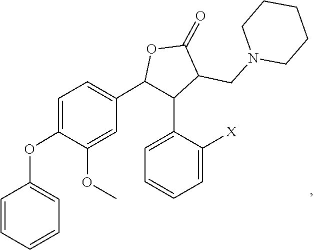

or a pharmaceutically acceptable salt thereof,
wherein X is a halo.

In embodiments, the disclosure relates to a compound for treating a patient with an infection having the formula

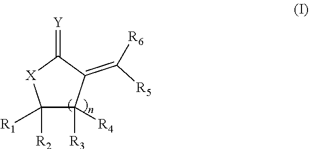

wherein:
   X is O, NH, NR, or S;
   Y is O, NH, NR, or S;
   n is 1, 2, 3, or 4;
   R is H or alkyl; and
   R₁, R₂, R₃, R₄, R₅, and R₆ are independently hydrogen, halogen, hydroxyl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted;
   or a pharmaceutically acceptable salt thereof.

In embodiments, the disclosure relates to a compound for treating a patient with an infection having the formula

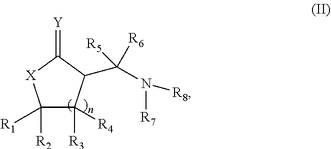

or a pharmaceutically acceptable salt thereof,
wherein
X is O, NH, NR, or S;
Y is O, NH, NR, or S;
n is 1, 2, 3, or 4;
R is H or alkyl;
R₁, R₂, R₃, R₄, R₅, and R₆ are independently hydrogen, halogen, hydroxyl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
R₇ and R₈ are independently hydrogen, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; or R₇ and R₈ are taken together to form a ring system with the N to which they are attached.

In embodiments, the disclosure relates to a compound for treating a patient with an infection having the formula

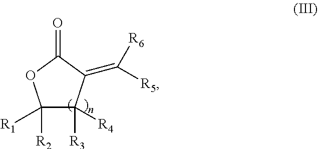

or a pharmaceutically acceptable salt thereof,
wherein
   n is 1, 2, 3, or 4; and
   R₁, R₂, R₃, R₄, R₅, and R₆ are independently hydrogen, halogen, hydroxyl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted.

In embodiments, the disclosure relates to a compound for treating a patient with an infection having the formula

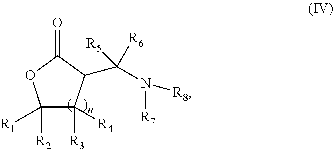

or a pharmaceutically acceptable salt thereof,
wherein
n is 1, 2, 3, or 4;
R₁, R₂, R₃, R₄, R₅, and R₆ are independently hydrogen, halogen, hydroxyl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
R₇ and R₈ are independently hydrogen, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; or R₇ and R₈ are taken together to form a ring system with the N to which they are attached.

In formula (I), (II), (III) or (IV) X can be O, X can be NH, X can be N(alkyl), X can be —N(Me), or X can be S.

In formula (I), (II), (III) or (IV) Y can be O, Y can be NH, Y can be N(alkyl), Y can be —N(Me), or Y can be S.

In formula (I), (II), (III) or (IV) n can be 1, n can be 2, n can be 3, or n can be 4.

R₁ can be hydrogen. R₁ can be halogen. R₁ can be hydroxyl. R₁ can be an optionally substituted alkyl. R₁ can be an optionally substituted alkenyl. R₁ can be an optionally substituted alkynyl. R₁ can be an optionally substituted heteroalkyl. R₁ can be an optionally substituted heteroalkenyl, R₁ can be an optionally substituted heteroalkynyl. R₁ can be an optionally substituted heterocyclyl. R₁ can be an optionally substituted cycloalkyl. R₁ can be an optionally substituted cycloalkenyl. R₁ can be an optionally substituted cycloheteroalkyl. R₁ can be an optionally substituted cycloheteroalkenyl. R₁ can be an acyl. R₁ can be an optionally substituted aryl. R₁ can be an optionally substituted heteroaryl. R₁ can be an optionally substituted arylalkyl. R₁ can be an optionally substituted arylalkenyl. R₁ can be an optionally substituted arylalkynyl.

R₂ can be hydrogen. R₂ can be halogen. R₂ can be hydroxyl. R₂ can be optionally substituted alkyl. R₂ can be optionally substituted alkenyl. R₂ can be optionally substituted alkynyl. R₂ can be optionally substituted heteroalkyl. R₂ can be optionally substituted heteroalkenyl. R₂ can be optionally substituted heteroalkynyl. R₂ can be optionally substituted heterocyclyl. R₂ can be optionally substituted cycloalkyl. R₂ can be optionally substituted cycloalkenyl. R₂ can be optionally substituted cycloheteroalkyl. R₂ can be optionally substituted cycloheteroalkenyl. R₂ can be acyl. R₂ can be optionally substituted aryl. R₂ can be optionally substituted heteroaryl. R₂ can be optionally substituted arylalkyl. R₂ can be optionally substituted arylalkenyl. R₂ can be optionally substituted arylalkynyl.

R₃ can be hydrogen. R₃ can be halogen. R₃ can be hydroxyl. R₃ can be optionally substituted alkyl. R₃ can be optionally substituted alkenyl. R₃ can be optionally substituted alkynyl. R₃ can be optionally substituted heteroalkyl. R₃ can be optionally substituted heteroalkenyl. R₃ can be optionally substituted heteroalkynyl. R₃ can be optionally substituted heterocyclyl. R₃ can be optionally substituted cycloalkyl. R₃ can be optionally substituted cycloalkenyl. R₃ can be optionally substituted cycloheteroalkyl. R₃ can be optionally substituted cycloheteroalkenyl. R₃ can be acyl. R₃ can be optionally substituted aryl. R₃ can be optionally substituted heteroaryl. R₃ can be optionally substituted arylalkyl. R₃ can be optionally substituted arylalkenyl. R₃ can be optionally substituted arylalkynyl.

R₄ can be hydrogen. R₄ can be halogen. R₄ can be hydroxyl. R₄ can be optionally substituted alkyl. R₄ can be optionally substituted alkenyl. R₄ can be optionally substituted alkynyl. R₄ can be optionally substituted heteroalkyl. R₄ can be optionally substituted heteroalkenyl. R₄ can be optionally substituted heteroalkynyl. R₄ can be optionally substituted heterocyclyl. R₄ can be optionally substituted cycloalkyl. R₄ can be optionally substituted cycloalkenyl. R₄ can be optionally substituted cycloheteroalkyl. R₄ can be optionally substituted cycloheteroalkenyl. R₄ can be acyl. R₄ can be optionally substituted aryl. R₄ can be optionally substituted heteroaryl. R₄ can be optionally substituted arylalkyl. R₄ can be optionally substituted arylalkenyl. R₄ can be optionally substituted arylalkynyl.

R₅ can be hydrogen. R₅ can be halogen. R₅ can be hydroxyl. R₅ can be optionally substituted alkyl. R₅ can be optionally substituted alkenyl. R₅ can be optionally substituted alkynyl. R₅ can be optionally substituted heteroalkyl. R₅ can be optionally substituted heteroalkenyl. R₅ can be optionally substituted heteroalkynyl. R₅ can be optionally substituted heterocyclyl. R₅ can be optionally substituted cycloalkyl. R₅ can be optionally substituted cycloalkenyl. R₅ can be optionally substituted cycloheteroalkyl. R₅ can be optionally substituted cycloheteroalkenyl. R₅ can be acyl. R₅ can be optionally substituted aryl. R₅ can be optionally substituted heteroaryl. R₅ can be optionally substituted arylalkyl. R₅ can be optionally substituted arylalkenyl. R₅ can be optionally substituted arylalkynyl.

R₆ can be hydrogen. R₆ can be halogen. R₆ can be hydroxyl. R₆ can be optionally substituted alkyl. R₆ can be optionally substituted alkenyl. R₆ can be optionally substituted alkynyl. R₆ can be optionally substituted heteroalkyl. R₆ can be optionally substituted heteroalkenyl. R₆ can be optionally substituted heteroalkynyl. R₆ can be optionally substituted heterocyclyl. R₆ can be optionally substituted cycloalkyl. R₆ can be optionally substituted cycloalkenyl. R₆ can be optionally substituted cycloheteroalkyl. R₆ can be optionally substituted cycloheteroalkenyl. R₆ can be acyl. R₆ can be optionally substituted aryl. R₆ can be optionally substituted heteroaryl. R₆ can be optionally substituted arylalkyl. R₆ can be optionally substituted arylalkenyl. R₆ can be optionally substituted arylalkynyl.

R₇ can be hydrogen. R₇ can be optionally substituted alkyl. R₇ can be optionally substituted alkenyl. R₇ can be optionally substituted alkynyl. R₇ can be optionally substituted heteroalkyl. R₇ can be optionally substituted heteroalkenyl. R₇ can be optionally substituted heteroalkynyl. R₇ can be optionally substituted heterocyclyl. R₇ can be optionally substituted cycloalkyl. R₇ can be optionally substituted cycloalkenyl. R₇ can be optionally substituted cycloheteroalkyl. R₇ can be optionally substituted cycloheteroalkenyl. R₇ can be acyl. R₇ can be optionally substituted aryl. R₇ can be optionally substituted heteroaryl. R₇ can be optionally substituted arylalkyl. R₇ can be optionally substituted arylalkenyl. R₇ can be optionally substituted arylalkynyl.

R₈ can be hydrogen. R₈ can be optionally substituted alkyl. R₈ can be optionally substituted alkenyl. R₈ can be optionally substituted alkynyl. R₈ can be optionally substituted heteroalkyl. R₈ can be optionally substituted heteroalkenyl. R₈ can be optionally substituted heteroalkynyl. R₈ can be optionally substituted heterocyclyl. R₈ can be optionally substituted cycloalkyl. R₈ can be optionally substituted cycloalkenyl. R₈ can be optionally substituted cycloheteroalkyl. R₈ can be optionally substituted cycloheteroalkenyl. R₈ can be acyl. R₈ can be optionally substituted aryl. R₈ can be optionally substituted heteroaryl. R₈ can be optionally substituted arylalkyl. R₈ can be optionally substituted arylalkenyl. R₈ can be optionally substituted arylalkynyl.

R₇ and R₈ can be taken together to form a ring system with the N to which they are attached. The ring system can comprise one or more additional heteroatom. The ring system can be a mono-, bi- or poly-cyclic ring system. The ring system can be substituted or unsubstituted. The ring system can be a 3-12 membered ring system.

In embodiments, the disclosure relates to a compound having the formula

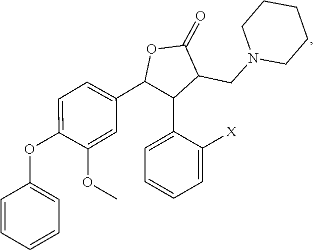

or a pharmaceutically acceptable salt thereof,
wherein X is a halo.

In embodiments, the disclosure relates to a compound for treating a patient with an infection having the formula

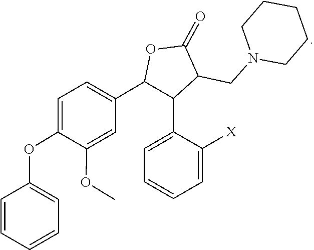

or a pharmaceutically acceptable salt thereof,
wherein X is a halo.

In embodiments, the infection is caused by Clostridioides difficile.

In embodiments, the disclosure relates to a compound selected from

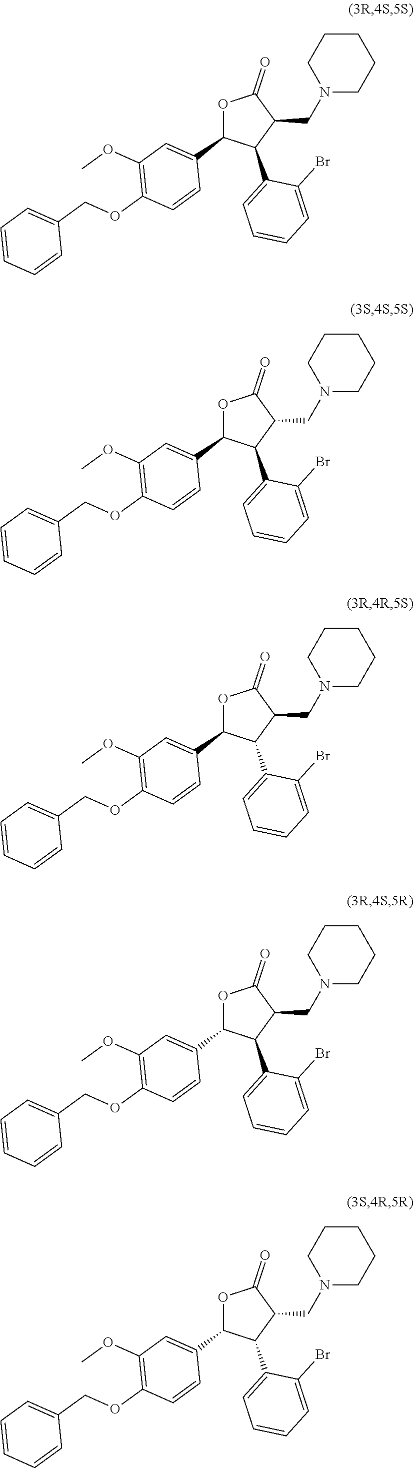 [SMILES: COc1cc([C@H]2OC(=O)[C@@H](CN3CCCCC3)[C@H]2c2ccccc2Br)ccc1OCc1ccccc1 ; COc1cc([C@H]2OC(=O)[C@H](CN3CCCCC3)[C@H]2c2ccccc2Br)ccc1OCc1ccccc1 ; COc1cc([C@H]2OC(=O)[C@@H](CN3CCCCC3)[C@@H]2c2ccccc2Br)ccc1OCc1ccccc1 ; COc1cc([C@@H]2OC(=O)[C@@H](CN3CCCCC3)[C@H]2c2ccccc2Br)ccc1OCc1ccccc1 ; COc1cc([C@@H]2OC(=O)[C@H](CN3CCCCC3)[C@@H]2c2ccccc2Br)ccc1OCc1ccccc1]

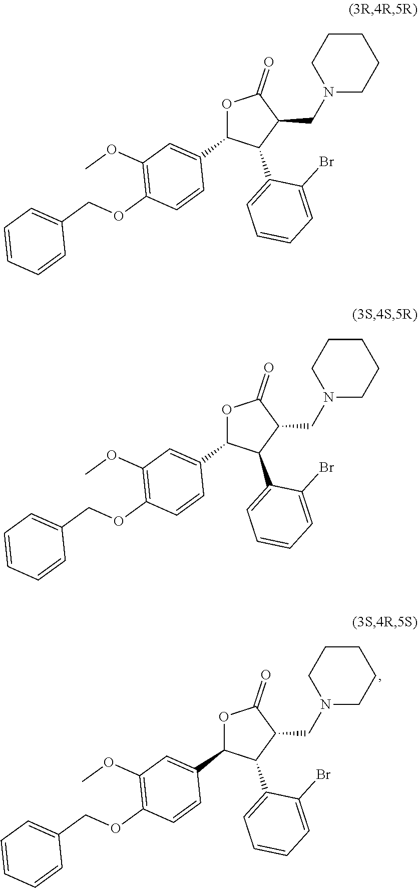 [SMILES: COc1cc([C@@H]2OC(=O)[C@@H](CN3CCCCC3)[C@@H]2c2ccccc2Br)ccc1OCc1ccccc1 ; COc1cc([C@@H]2OC(=O)[C@H](CN3CCCCC3)[C@H]2c2ccccc2Br)ccc1OCc1ccccc1 ; COc1cc([C@H]2OC(=O)[C@H](CN3CCCCC3)[C@@H]2c2ccccc2Br)ccc1OCc1ccccc1]

or a pharmaceutically acceptable salt thereof.

In embodiments, the disclosure relates to a compound for treating a patient with an infection having the formula

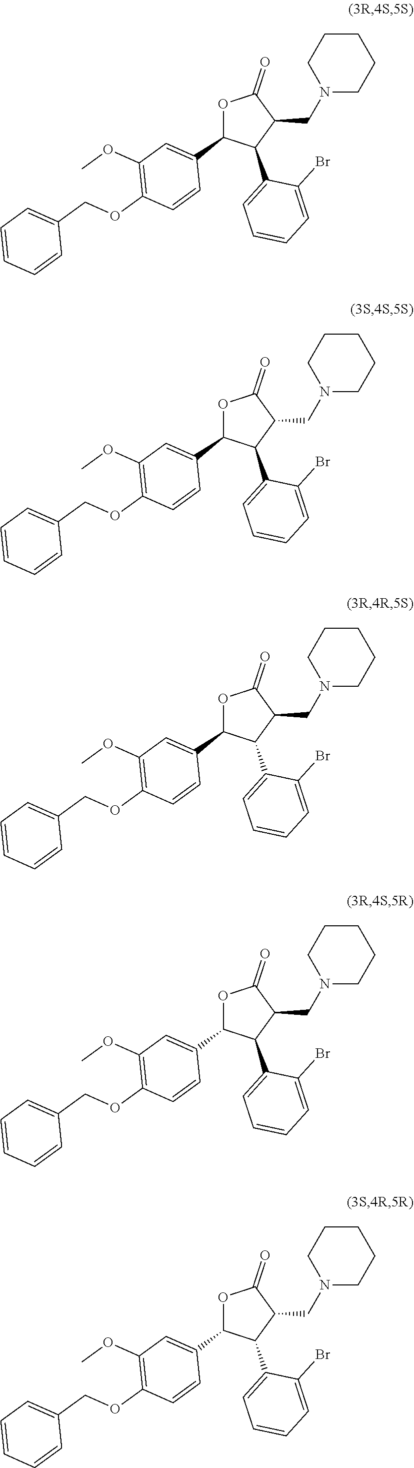 [SMILES: COc1cc([C@H]2OC(=O)[C@@H](CN3CCCCC3)[C@H]2c2ccccc2Br)ccc1OCc1ccccc1 ; COc1cc([C@H]2OC(=O)[C@H](CN3CCCCC3)[C@H]2c2ccccc2Br)ccc1OCc1ccccc1 ; COc1cc([C@H]2OC(=O)[C@@H](CN3CCCCC3)[C@@H]2c2ccccc2Br)ccc1OCc1ccccc1 ; COc1cc([C@@H]2OC(=O)[C@@H](CN3CCCCC3)[C@H]2c2ccccc2Br)ccc1OCc1ccccc1 ; COc1cc([C@@H]2OC(=O)[C@H](CN3CCCCC3)[C@@H]2c2ccccc2Br)ccc1OCc1ccccc1]

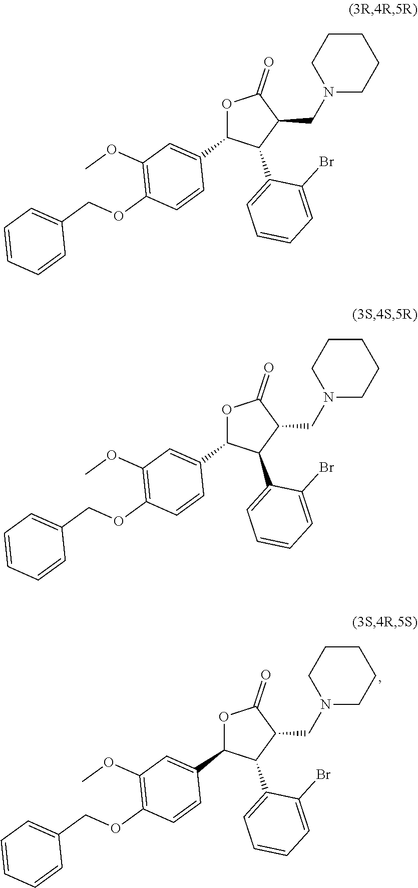 [SMILES: COc1cc([C@@H]2OC(=O)[C@@H](CN3CCCCC3)[C@@H]2c2ccccc2Br)ccc1OCc1ccccc1 ; COc1cc([C@@H]2OC(=O)[C@H](CN3CCCCC3)[C@H]2c2ccccc2Br)ccc1OCc1ccccc1 ; COc1cc([C@H]2OC(=O)[C@H](CN3CCCCC3)[C@@H]2c2ccccc2Br)ccc1OCc1ccccc1]

or a pharmaceutically acceptable salt thereof,
wherein X is a halo.

In embodiments, the infection is caused by Clostridioides difficile.

In embodiments, the disclosure relates to a pharmaceutical composition comprising one or more compounds, together with one or more pharmaceutically acceptable diluents, excipients or carriers.

In embodiments, the disclosure relates to a pharmaceutical composition comprising one or more compounds of formula (I), (II), (III), or (IV), together with one or more diluents, excipients or carriers.

In embodiments, the disclosure relates to a pharmaceutical composition comprising nanoparticles of one or more compounds of formula (I), (II), (III), or (IV), together with one or more diluents, excipients or carriers.

In embodiments, the disclosure relates to the use of one or more compounds or a pharmaceutically acceptable salt thereof in the manufacture of a medicament for treating Clostridioides difficile infection in a subject.

In embodiments, the disclosure relates to a method of use of a compound or a pharmaceutically acceptable salt thereof in the manufacture of a medicament for treating infection in a subject.

As used herein, the following terms and phrases shall have the meanings set forth below. Unless defined otherwise, all technical and scientific terms used herein have the same meaning as commonly understood to one of ordinary skill in the art.

The term “about” can allow for a degree of variability in a value or a range, for example, within 10%, within 5%, or within 1% of a stated value or of a stated limit of a range. The term “substantially” can allow for a degree of variability in a value or a range, for example, within 90%, within 95%, 99%, 99.5%, 99.9%, 99.99%, or 99.999% or more of a stated value or of a stated limit of a range.

The terms “a,” “an,” or “the” are used to include one or more than one unless the context clearly dictates otherwise. The term “or” is used to refer to a nonexclusive “or” unless otherwise indicated.

The phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section.

All publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

The term “substituted” refers to a functional group in which one or more hydrogen atoms contained therein are replaced by one or more non-hydrogen atoms. The term “functional group” or “substituent” refers to a group that can be or is substituted onto a molecule. Examples of substituents or functional groups include, but are not limited to, a halogen (e.g., F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, azides, hydroxylamines, cyano, nitro groups, N-oxides, hydrazides, and enamines; and other heteroatoms in various other groups. It will be understood that “substitution” or “substituted with” includes the implicit proviso that such substitution is in accordance with permitted valence of the substituted atom and the substituent, and that the substitution results in a stable compound, e.g., a compound which does not spontaneously undergo transformation such as by rearrangement, cyclization, elimination, etc. The term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. Heteroatoms, such as nitrogen, may have hydrogen substituents and/or any permissible substituents of organic compounds which satisfy the valences of the heteroatoms. Substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aryl, or an aromatic or heteroaromatic moiety. The substituents on substituted alkyls can be selected from C₁₋₆ alkyl, C₃₋₆ cycloalkyl, halogen, carbonyl, cyano, or hydroxyl. The substituents on substituted alkyls can be selected from fluoro, carbonyl, cyano, or hydroxyl. It will be understood by those skilled in the art that substituents can themselves be substituted, if appropriate. Unless specifically stated as “unsubstituted,” references to chemical moieties include substituted variants. For example, reference to an “aryl” group or moiety implicitly includes both substituted and unsubstituted variants.

The term “substituted” also refers to a group that is substituted with one or more groups including, but not limited to, the following groups: halogen (e.g., F, Cl, Br, and I), R, OR, ROH (e.g., CH₂OH), OC(O)N(R)₂, CN, NO, NO₂, ONO₂, azido, CF₃, OCF₃, methylenedioxy, ethylenedioxy, (C₃-C₂₀)heteroaryl, N(R)₂, Si(R)₃, SR, SOR, SO₂R, SO₂N(R)₂, SO₃R, P(O)(OR)₂, OP(O)(OR)₂, C(O)R, C(O)C(O)R, C(O)CH₂C(O)R, C(S)R, C(O)OR, OC(O)R, C(O)N(R)₂, C(O)N(R)OH, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, or C(═NOR)R wherein R can be hydrogen, (C₁-C₂₀)alkyl, (C₆-C₂₀)aryl, heterocyclyl or polyalkylene oxide groups, such as polyalkylene oxide groups of the formula —(CH₂CH₂O)_{f}—R—OR, —(CH₂CH₂CH₂O)_{g}—R—OR, or —(CH₂CH₂O)_{f}(CH₂CH₂CH₂O)_{g}—R—OR, each of which can, in turn, be substituted or unsubstituted and wherein f and g are each independently an integer from 1 to 50 (e.g., 1 to 3, 1 to 5, 1 to 10, 1 to 20, 1 to 30, 1 to 40, 1-50, 2 to 5, 2 to 10, 2 to 20, 2 to 30, 2 to 40, 2 to 50, 3 to 10, 3 to 20, 3 to 40, 3 to 50, 5 to 10, 5 to 20, 5 to 30, 5 to 40, 5-50, and any other ranges in between). “Substituted” also includes a group that is substituted with one or more groups including, but not limited to, the following groups: fluoro, chloro, bromo, iodo, amino, amido, alkyl, hydroxy, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. Where there are two or more adjacent substituents, the substituents can be linked to form a carbocyclic or heterocyclic ring. Such adjacent groups can have a vicinal or germinal relationship, or they can be adjacent on a ring in, e.g., an ortho-arrangement. Each instance of “substituted” is understood to be independent. For example, a substituted aryl can be substituted with bromo and a substituted heterocycle on the same compound can be substituted with alkyl. It is envisaged that a substituted group can be substituted with one or more non-fluoro groups. As another example, a substituted group can be substituted with one or more non-cyano groups. As another example, a substituted group can be substituted with one or more groups other than haloalkyl. As yet another example, a substituted group can be substituted with one or more groups other than tert-butyl. As yet a further example, a substituted group can be substituted with one or more groups other than trifluoromethyl. As yet even further examples, a substituted group can be substituted with one or more groups other than nitro, other than methyl, other than methoxymethyl, other than dialkylaminosulfonyl, other than bromo, other than chloro, other than amido, other than halo, other than benzodioxepinyl, other than polycyclic heterocyclyl, other than polycyclic substituted aryl, other than methoxycarbonyl, other than alkoxycarbonyl, other than thiophenyl, or other than nitrophenyl, or groups meeting a combination of such descriptions. Further, “substituted” is also understood to include fluoro, cyano, haloalkyl, tert-butyl, trifluoromethyl, nitro, methyl, methoxymethyl, dialkylaminosulfonyl, bromo, chloro, amido, halo, benzodioxepinyl, polycyclic heterocyclyl, polycyclic substituted aryl, methoxycarbonyl, alkoxycarbonyl, thiophenyl, and nitrophenyl groups.

“Alkyl” refers to a fully saturated cyclic or acyclic, branched or unbranched carbon chain moiety having the number of carbon atoms specified, or up to 30 carbon atoms if no specification is made. For example, an alkyl of 1 to 8 carbon atoms refers to moieties such as methyl, ethyl, propyl, butyl, pentyl, hexyl, heptyl, and octyl, and those moieties which are positional isomers of these moieties. Alkyl of 10 to 30 carbon atoms includes decyl, undecyl, dodecyl, tridecyl, tetradecyl, pentadecyl, hexadecyl, heptadecyl, octadecyl, nonadecyl, eicosyl, heneicosyl, docosyl, tricosyl and tetracosyl. A straight chain or branched chain alkyl can have 30 or fewer carbon atoms in its backbone (e.g., C₁-C₃₀ for straight chains, C₃-C₃₀ for branched chains), or 20 or fewer. Alkyl groups may be substituted or unsubstituted.

The term “alkylene” refers to an alkyl group having the specified number of carbons, for example from 2 to 12 carbon atoms, that contain two points of attachment to the rest of the compound on its longest carbon chain. Non-limiting examples of alkylene groups include methylene —(CH₂)—, ethylene —(CH₂CH₂)—, n-propylene —(CH₂CH₂CH₂)—, isopropylene —(CH₂CH(CH₃))—, and the like. Alkylene groups can be cyclic or acyclic, branched or unbranched carbon chain moieties and may be optionally substituted with one or more substituents. For example, alkylene-aryl can be benzyl.

“Cycloalkyl” means mono- or bicyclic or bridged or spirocyclic, or polycyclic saturated carbocyclic rings, each having from 3 to 12 carbon atoms. In various aspects, cycloalkyls have from 3-10 carbon atoms in their ring structure, or 3-6 carbons in the ring structure. Cycloalkyl groups may be substituted or unsubstituted. Examples of cycloalkyl include cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms can range from 3 to 4, 5, 6, or 7. In some embodiments, cycloalkyl groups can have 3 to 6 carbon atoms (C₃-C₆). Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like

Unless the number of carbons is otherwise specified, “lower alkyl,” as used herein, means an alkyl group, as defined above, but having from one to ten carbons, or from one to six carbon atoms in its backbone structure such as methyl, ethyl, n-propyl, isopropyl, n-butyl, isobutyl, sec-butyl, and tert-butyl. Likewise, “lower alkenyl” and “lower alkynyl” have similar chain lengths. A substituent designated herein as alkyl can be a lower alkyl.

“Alkenyl” refers to any cyclic or acyclic, branched or unbranched unsaturated carbon chain moiety having the number of carbon atoms specified, or up to 26 carbon atoms if no limitation on the number of carbon atoms is specified; and having one or more double bonds in the moiety. Alkenyl of 6 to 26 carbon atoms is exemplified by hexenyl, heptenyl, octenyl, nonenyl, decenyl, undecenyl, dodenyl, tridecenyl, tetradecenyl, pentadecenyl, hexadecenyl, heptadecenyl, octadecenyl, nonadecenyl, eicosenyl, heneicosoenyl, docosenyl, tricosenyl, and tetracosenyl, in their various isomeric forms, where the unsaturated bond(s) can be located anywhere in the moiety and can have either the (Z) or the (E) configuration about the double bond(s).

“Alkynyl” refers to hydrocarbyl moieties of the scope of alkenyl but having one or more triple bonds in the moiety.

The term “acyl” refers to a group containing a carbonyl moiety wherein the group is bonded via the carbonyl carbon atom. The carbonyl carbon atom is also bonded to another carbon atom, which can be part of a substituted or unsubstituted alkyl, aryl, aralkyl cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-40, 6-10, 1-5 or 2-5 additional carbon atoms bonded to the carbonyl group. An acryloyl group is an example of an acyl group. An acyl group can also include heteroatoms within the meaning here. A nicotinoyl group (pyridyl-3-carbonyl) is an example of an acyl group. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “aryl” includes 3- to 12-membered substituted or unsubstituted single-ring aromatic groups in which each atom of the ring is carbon (i.e., carbocyclic aryl) or where one or more atoms are heteroatoms (i.e., heteroaryl). In various aspects, aryl groups include 5- to 12-membered rings, or 6- to 10-membered rings. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Carbocyclic aryl groups include benzene, naphthalene, phenanthrene, phenol, aniline, and the like. Heteroaryl groups include substituted or unsubstituted aromatic 3- to 12-membered ring structures, 5- to 12-membered rings, or 5- to 10-membered rings, whose ring structures include one to four heteroatoms. Heteroaryl groups include, for example, pyrrole, furan, thiophene, imidazole, oxazole, thiazole, triazole, pyrazole, pyridine, pyrazine, pyridazine and pyrimidine, and the like. Aryl and heteroaryl can be monocyclic, bicyclic, or polycyclic. Each instance of an aryl group may be independently optionally substituted, i.e., unsubstituted (an “unsubstituted aryl”) or substituted (a “substituted aryl”) with one or more substituents; e.g., for instance from 1 to 5 substituents, 1 to 4 substituents, 1 to 3 substituents, 1 to 2 substituents or just 1 substituent. The aromatic ring may be substituted at one or more ring positions with one or more substituents, such as halogen, azide, alkyl, aryl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluromethyl), cyano, or the like. For example, the aryl group can be an unsubstituted C₅-C₁₂ aryl or the aryl group can be a substituted C₅-C₁₀ aryl. Aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons (C₆-C₁₄) or from 6 to 10 carbon atoms (C₆-C₁₀) in the ring portions of the groups. Aryl groups can be unsubstituted or substituted, as defined herein. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed herein.

The terms “aralkyl” and “arylalkyl” refer to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The terms “heterocyclyl” and “heterocyclic group” refer to 3- to 12-membered ring structures, 5- to 12-membered rings, or 5- to 10-membered rings, whose ring structures include one to four heteroatoms. Heterocycles can be monocyclic, bicyclic, spirocyclic, or polycyclic. Heterocycles can be saturated or unsaturated. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, for example, halogen, alkyl, aryl, alkenyl, alkynyl, cycloalkyl, hydroxyl, amino, nitro, sulfhydryl, imino, amido, phosphate, phosphonate, phosphinate, carbonyl, carboxyl, silyl, sulfamoyl, sulfinyl, ether, alkylthio, sulfonyl, ketone, aldehyde, ester, a heterocyclyl, an aromatic or heteroaromatic moiety, —CF₃, —CN, and the like.

A heteroaryl ring is an embodiment of a heterocyclyl group. The phrase “heterocyclyl group” includes fused ring species including those that include fused aromatic and non-aromatic groups. Representative heterocyclyl groups include, but are not limited to, piperidynyl, piperazinyl, morpholinyl, furanyl, pyrrolidinyl, pyridinyl, pyrazinyl, pyrimidinyl, triazinyl, thiophenyl, tetrahydrofuranyl, pyrrolyl, oxazolyl, imidazolyl, triazyolyl, tetrazolyl, benzoxazolinyl, and benzimidazolinyl groups. For example, heterocyclyl groups include, without limitation:

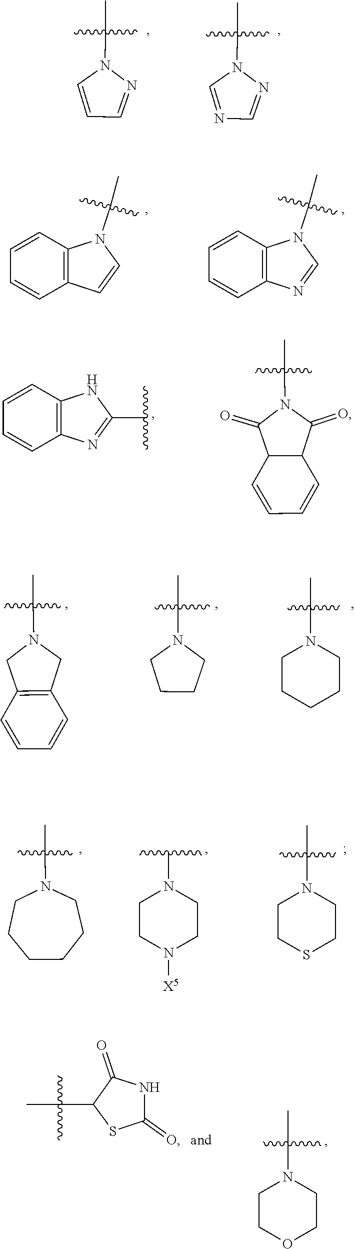

wherein X⁵ represents H, (C₁-C₂₀)alkyl, (C₆-C₂₀)aryl or an amine protecting group (e.g., a t-butyloxycarbonyl group) and wherein the heterocyclyl group can be substituted or unsubstituted. A nitrogen-containing heterocyclyl group is a heterocyclyl group containing a nitrogen atom as an atom in the ring. In some embodiments, the heterocyclyl is other than thiophene or substituted thiophene. In some embodiments, the heterocyclyl is other than furan or substituted furan.

A heteroaryl ring is an embodiment of a heterocyclyl group. The phrase “heterocyclyl group” includes fused ring species including those that include fused aromatic and non-aromatic groups. Representative heterocyclyl groups include, but are not limited to pyrrolidinyl, azetidinyl, piperidynyl, piperazinyl, morpholinyl, chromanyl, indolinonyl, isoindolinonyl, furanyl, pyrrolidinyl, pyridinyl, pyrazinyl, pyrimidinyl, triazinyl, thiophenyl, tetrahydrofuranyl, pyrrolyl, oxazolyl, oxadiazolyl, imidazolyl, triazyolyl, tetrazolyl, benzoxazolinyl, benzthiazolinyl, and benzimidazolinyl groups.

The term “heterocyclylalkyl” refers to alkyl groups as defined herein in which a hydrogen or carbon bond of an alkyl group as defined herein is replaced with a bond to a heterocyclyl group as defined herein. Representative heterocyclylalkyl groups include, but are not limited to, furan-2-yl methyl, furan-3-yl methyl, pyridine-3-yl methyl, tetrahydrofuran-2-yl methyl, and indol-2-yl propyl.

The term “heteroarylalkyl” refers to alkyl groups as defined herein in which a hydrogen or carbon bond of an alkyl group is replaced with a bond to a heteroaryl group as defined herein.

The term “alkoxy” refers to an oxygen atom connected to an alkyl group, including a cycloalkyl group, as defined herein. Examples of linear alkoxy groups include, but are not limited to, methoxy, ethoxy, propoxy, butoxy, pentyloxy, hexyloxy, and the like. Examples of branched alkoxy include, but are not limited to, isopropoxy, sec-butoxy, tert-butoxy, isopentyloxy, isohexyloxy, and the like. Examples of cyclic alkoxy include, but are not limited to, cyclopropyloxy, cyclobutyloxy, cyclopentyloxy, cyclohexyloxy, and the like. An alkoxy group can further include double or triple bonds, and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The term “amine” refers to primary, secondary, and tertiary amines having, e.g., the formula N(group)₃ wherein each group can independently be H or non-H, such as alkyl, aryl, and the like. Amines include, but are not limited to, R—NH₂, for example, alkylamines, arylamines, alkylarylamines; R₂NH, wherein each R is independently selected, such as dialkylamines, diarylamines, aralkylamines, heterocyclylamines and the like; and R₃N, wherein each R is independently selected, such as trialkylamines, dialkylarylamines, alkyldiarylamines, triarylamines, and the like. The term “amine” also includes ammonium ions as used herein.

The term “amino group” refers to a substituent of the form —NH₂, —NHR, —NR₂, or —NR₃⁺ wherein each R is independently selected, and protonated forms of each, except for —NR₃⁺, which cannot be protonated. Accordingly, any compound substituted with an amino group can be viewed as an amine. An “amino group” can be a primary, secondary, tertiary, or quaternary amino group. An “alkylamino” group includes a monoalkylamino, dialkylamino, and trialkylamino group. The term “halo”, “halide”, or “halogen” means halogen and includes, for example, and without being limited thereto, fluoro, chloro, bromo, iodo and the like, in both radioactive and non-radioactive forms. Halo can be selected from the group consisting of fluoro, chloro and bromo.

The term “haloalkyl” includes mono-halo alkyl groups, poly-halo alkyl groups, wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, —CF(CH₃)₂ and the like.

The term “optionally substituted” or “optional substituents” means that the groups in question are either unsubstituted or substituted with one or more of the substituents specified. When the groups in question are substituted with more than one substituent, the substituents may be the same or different. The terms “independently,” “independently are,” and “independently selected from” mean that the groups in question may be the same or different. Certain of the herein defined terms may occur more than once in the structure, and upon such occurrence each term shall be defined independently of the other.

The term “nitro” means —NO₂; the term “halogen” designates —F, —Cl, —Br, or —I; the term “sulfhydryl” means —SH; the term “hydroxyl” means —OH; the term “sulfonyl” means —SO₂—; the term “azido” means —N3; the term “cyano” means —CN; the term “isocyanato” means —NCO; the term “thiocyanato” means —SCN; the term “isothiocyanato” means —NCS; and the term “cyanato” means —OCN.

The compounds may contain one or more chiral centers or may otherwise be capable of existing as multiple stereoisomers. In one embodiment, the compounds are not limited to any particular stereochemical requirement, and that the compounds, and compositions, methods, uses, and medicaments that include them may be optically pure, or may be any of a variety of stereoisomeric mixtures, including racemic and other mixtures of enantiomers, other mixtures of diastereomers, and the like. Such mixtures of stereoisomers may include a single stereochemical configuration at one or more chiral centers, while including mixtures of stereochemical configuration at one or more other chiral centers.

Similarly, the compounds may include geometric centers, such as cis, trans isomers, diastereomers, enantiomers, and E and Z double bonds. In another embodiment, the compounds are not limited to any particular geometric isomer requirement, and that the compounds, and compositions, methods, uses, and medicaments that include them may be pure, or may be any of a variety of geometric isomer mixtures. Such mixtures of geometric isomers may include a single configuration at one or more double bonds and chiral carbons, while including mixtures of geometry at one or more other double bonds and chiral carbons.

The terms “salts” and “pharmaceutically acceptable salts” refer to derivatives of the compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound, which contains a basic or acidic moiety, by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference for its teachings regarding same.

The term “solvate” means a compound, or a salt thereof, that further includes a stoichiometric or non-stoichiometric amount of solvent bound by non-covalent intermolecular forces. Where the solvent is water, the solvate is a hydrate.

The term “prodrug” means a derivative of a compound that can hydrolyze, oxidize, or otherwise react under biological conditions (in vitro or in vivo) to provide an active compound of the present disclosure. Examples of prodrugs include, but are not limited to, derivatives and metabolites of a compound that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Specific prodrugs of compounds with carboxyl functional groups are the lower alkyl esters of the carboxylic acid. The carboxylate esters are conveniently formed by esterifying any of the carboxylic acid moieties present on the compound. Prodrugs can typically be prepared using well-known methods, such as those described by Burger's Medicinal Chemistry and Drug Discovery 6th ed. (Donald J. Abraham ed., 2001, Wiley) and Design and Application of Prodrugs (H. Bundgaard ed., 1985, Harwood Academic Publishers GmbH), both of which are hereby incorporated by reference for their teachings regarding same.

Further, in each of the foregoing and following embodiments, it is to be understood that the formulae include and represent not only all pharmaceutically acceptable salts of the compounds, but also include any and all hydrates and/or solvates of the compound formulae or salts thereof. It is to be appreciated that certain functional groups, such as the hydroxy, amino, and like groups form complexes and/or coordination compounds with water and/or various solvents, in the various physical forms of the compounds. Accordingly, the above formulae are to be understood to include and represent those various hydrates and/or solvates. In each of the foregoing and following embodiments, it is also to be understood that the formulae include and represent each possible isomer, such as stereoisomers and geometric isomers, both individually and in any and all possible mixtures. In each of the foregoing and following embodiments, it is also to be understood that the formulae include and represent any and all crystalline forms, partially crystalline forms, and non-crystalline and/or amorphous forms of the compounds.

The term “pharmaceutically acceptable carrier” is art-recognized and refers to a pharmaceutically acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically acceptable salts” refers to the relatively non-toxic, inorganic and organic acid addition salts of the compound(s). These salts can be prepared in situ during the final isolation and purification of the compound(s), or by separately reacting a purified compound(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts, and the like. (See, for example, Berge et al. (1977) “Pharmaceutical Salts”, J. Pharm. Sci. 66:1-19, which is hereby incorporated by reference for its teachings regarding same.)

In other cases, the compounds useful in the methods of the present invention may contain one or more acidic functional groups and, thus, can form pharmaceutically acceptable salts with pharmaceutically acceptable bases. The term “pharmaceutically acceptable salts” in these instances refers to the relatively non-toxic inorganic and organic base addition salts of a compound(s). These salts can likewise be prepared in situ during the final isolation and purification of the compound(s), or by separately reacting the purified compound(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

A “therapeutically effective amount” (or “effective amount”) of a compound with respect to use in treatment, refers to an amount of the compound in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment. The therapeutic agent(s), including specifically but not limited to a compound of the invention, may be provided in particles. Particles as used herein means nanoparticles or microparticles (or in some instances larger particles) which can consist in whole or in part of the compound of the invention or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero-order release, first-order release, second-order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the compound of the invention in a solution or in a semi-solid state. The particles may be of virtually any shape.

The term “administering” includes all means of introducing the compounds and compositions to the patient, including, but not limited to, oral (po), intravenous (iv), intramuscular (im), subcutaneous (sc), transdermal, inhalation, buccal, ocular, sublingual, vaginal, rectal, and the like. The compounds and compositions may be administered in unit dosage forms and/or formulations containing conventional nontoxic pharmaceutically acceptable carriers, adjuvants, and vehicles.

Illustrative formats for oral administration include tablets, capsules, elixirs, syrups, and the like. Illustrative routes for parenteral administration include intravenous, intraarterial, intraperitoneal, epidural, intraurethral, intrasternal, intramuscular and subcutaneous, as well as any other art-recognized route of parenteral administration.

Illustrative means of parenteral administration include needle (including microneedle) injectors, needle-free injectors and infusion techniques, as well as any other means of parenteral administration recognized in the art. Parenteral formulations are typically aqueous solutions which may contain excipients such as salts, carbohydrates and buffering agents (preferably at a pH in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well-known to those skilled in the art. Parenteral administration of a compound is illustratively performed in the form of saline solutions or with the compound incorporated into liposomes. In cases where the compound is not sufficiently soluble to be dissolved, a solubilizer such as ethanol can be applied.

The dosage of each compound depends on several factors, including the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

In the methods the individual components of a co-administration or a combination can be administered by any suitable means, contemporaneously, simultaneously, sequentially (in either order), separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the therapy, concurrently in divided or single forms.

The term “therapeutically effective amount” refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, the total daily usage of the compounds and compositions may be decided by the attending physician within the scope of sound medical judgment. The specific therapeutically effective dose level for any particular patient will depend upon a variety of factors, including the disorder being treated and the severity of the disorder; activity of the specific compound employed; the specific composition employed; the age, body weight, general health, gender and diet of the patient; the time of administration, route of administration, and rate of excretion of the specific compound employed; the duration of the treatment; drugs used in combination or coincidentally with the specific compound employed; and like factors well-known to the researcher, veterinarian, medical doctor or other clinician of ordinary skill.

Depending upon the route of administration, a wide range of permissible dosages are contemplated, including doses falling in the range from about 1 g/kg to about 1 g/kg. The dosages may be single or divided and may be administered according to a wide variety of protocols, including q.d. (once a day), b.i.d. (twice a day), t.i.d. (three times a day), or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

In addition to the illustrative dosages and dosing protocols described herein, it is to be understood that an effective amount of any one or a mixture of the compounds can be determined by the attending diagnostician or physician using known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The term “patient” includes human and non-human animals such as companion animals (dogs and cats and the like) and livestock animals. Livestock animals are animals raised for food production. The patient to be treated is preferably a mammal, in particular a human being.

The preparative procedure of AAMGBLs (alpha-aminomethyl-gamma-butyrolactones) is described in Scheme 1.

A series of γ-hydroxy-α-methylene esters of the general structural formula V were prepared according to published procedures [21-25]. They were converted to the corresponding alpha-aminomethyl-γ-butyrolactones AMGBL (general structural formula VII) as described earlier. Molecules of formula VII were converted to the corresponding α-aminomethyllactones (general structural formula VI) as described by others [16,17] and us [26]. Molecules of formula I were directly converted to molecules VI as described by us [27].

The synthesis is shown in Scheme 1.

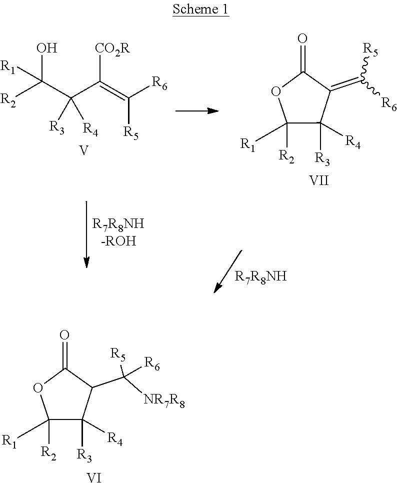

Provided herein is an alternate purification process to obtain AAMGBLs in extremely high purity. In the current invention, α-aminomethyllactones (general structural formula III), prepared from molecules of formula I and II, were purified by converting them to the corresponding fumarate salts, separating them from other impurities and adding base (NaOH).

The aminolactones can also be purified by the precipitation of organic or inorganic acid (e.g., HCl, HBr) or organic or inorganic salts (ammonium chloride or ammonium bromide) or organic acids salts, (e.g., oxalic acid, maleic acid, etc.) and treatment with base (e.g., LiOH, NaOH, or KOH).

This purification process allowed for a one-pot direct synthesis of α-aminomethyl-γ-butyrolactones (AAMGBLs) from carbonyls. A representative one-pot synthesis and purification for (cis)-5-(4-(benzyloxy)-3-methoxyphenyl)-4-(2-bromophenyl)-3-(piperidin-1-ylmethyl)dihydrofuran-2(3H)-one is shown in Scheme 2.

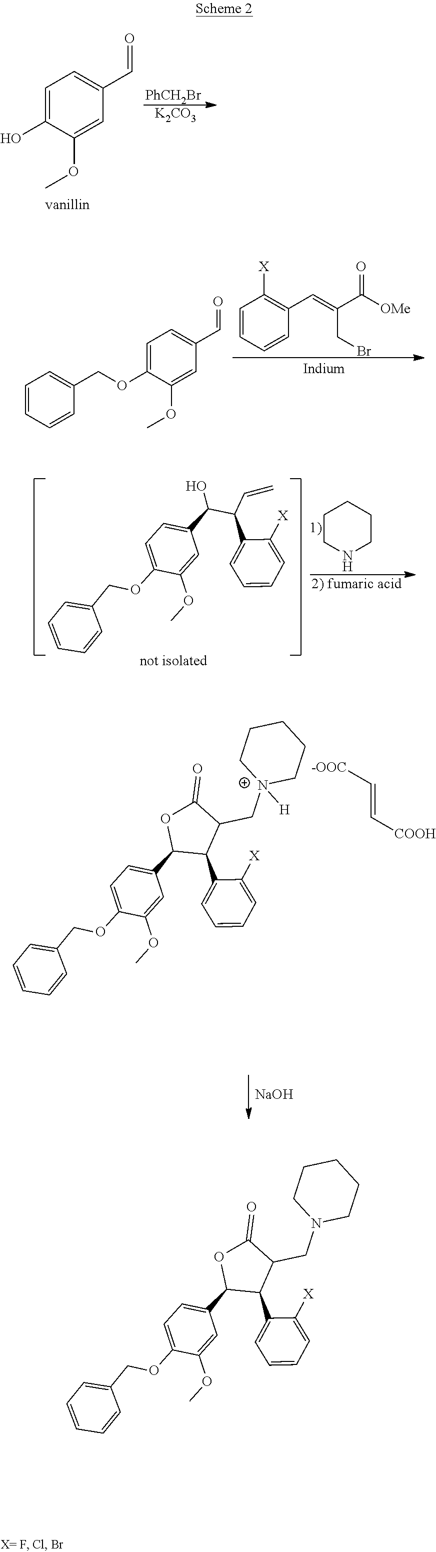

Representative procedures for the synthesis of compounds of formulae V, VI, and VII are given below.

### 1. General Procedure for Preparation of Cis-γ-Hydroxyester (Compound Formula V) [21].

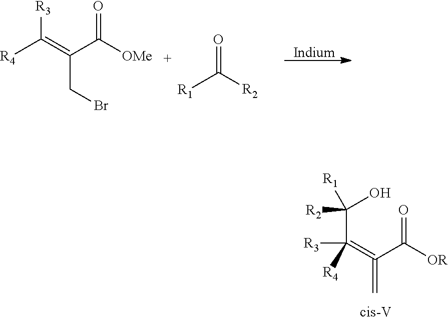

An appropriate aldehyde or ketone (1.5 equiv) was added to a solution of alkyl bromomethacrylate (1.0 eq) in a mixture of tetrahydrofuran (THF) and water. The solution was rapidly stirred at room temperature as indium powder (1.2 equiv) was added in one portion. The suspension was stirred until the reaction was complete as judged by thin layer chromatographic (TLC) analysis. The mixture was partitioned between water and ethyl acetate, and the organic layer was removed. The crude product was extracted from the aqueous layer one further time with ethyl acetate. The combined organic layers were washed with water and then brine, and then dried over sodium sulfate (Na₂SO₄). After filtration, the product was purified via flash column chromatography to yield the desired cis-γ-hydroxyester.

### 2. General Procedure for the Preparation of Trans-Lactone (Compound Formula VII).

### (i) General Procedure for the Preparation of Boronate [22].

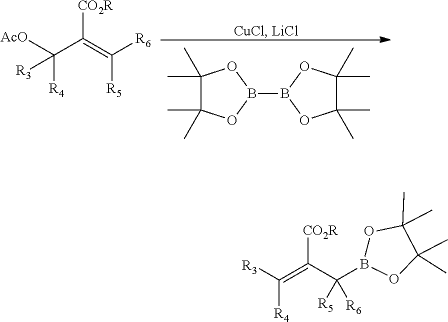

A mixture of CuCl (1.3 equiv) and LiCl (1.3 equiv) in dimethylformamide (DMF) was stirred under N₂ for 1 hour at 25° C., and pinacol diboronate (1.3 equiv) dissolved in DMF was added to the reaction mixture. Potassium acetate (KOAc) (1.3 equiv) was added to the reaction mixture, followed by the addition of allylic acetate (1.0 equiv) dissolved in DMF. The reaction mixture was further stirred for 3-5 hours. The reaction mixture was then quenched with water and extracted with ether. The combined organic layers were dried over magnesium sulfate (MgSO₄), concentrated under vacuum, and purified by column chromatography to obtain crotylboronates.

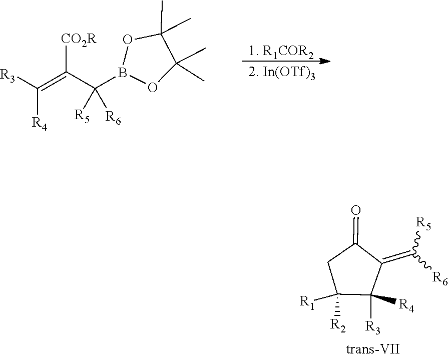

### (ii) General Procedure for the Preparation of Trans-Lactones [23].

Crotylboronate (1 equiv) was dissolved in toluene, and aldehyde (1.2 equiv) was added. The reaction mixture was refluxed for 8-12 hours until completion as was monitored by ¹¹B NMR spectroscopy. The reaction mixture was then cooled to room temperature, and 20% indium triflate [In(OTf)₃] was added, along with 20% aldehyde, and stirred for 2-4 hours. The mixture was washed with water and the product was extracted with ether (3×5 mL). The combined organic layer was washed with brine, dried over anhydrous MgSO₄, and concentrated in vacuo. The crude product was purified by column chromatography to obtain the trans-lactones.

### 3. Alternative Procedure for the Preparation of Trans-Lactone (Compound Formula VII) [24].

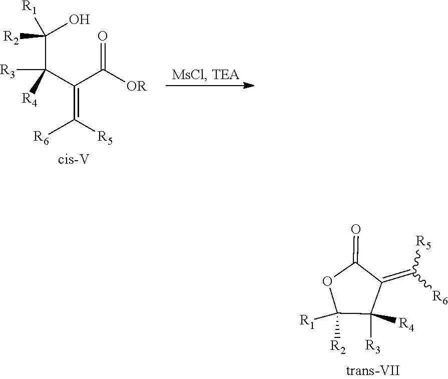

Triethylamine (1.1 equiv) was added to a solution of γ-hydroxyester (1.0 equiv) in dichloromethane (DCM). Methanesulfonyl chloride (1.1 equiv) was added dropwise, and the reaction mixture was stirred until judged complete by TLC analysis. The reaction mixture was quenched with saturated, aqueous ammonium sulfate, and then extracted with DCM. After concentrating the combined organic layers in vacuo, the product was purified via column chromatography to obtain the trans-lactone.

### 4. General Procedure for the Preparation of Cis-Lactone (Compound Formula VII) [25].

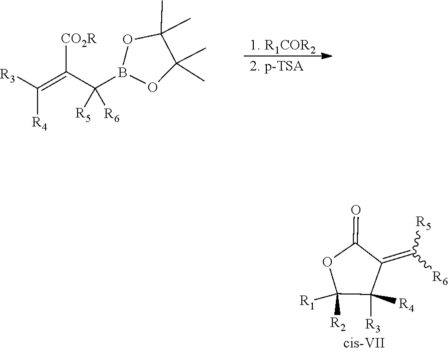

Crotylboronate (1 equiv) was dissolved in toluene, and aldehyde (1.2 equiv) was added. The reaction mixture was refluxed for 8-12 hours until completion as was monitored by ¹¹B NMR spectroscopy. The reaction mixture was then cooled to room temperature, and 20% p-TSA was added, along with 20% aldehyde, and stirred for 4-5 hours. The mixture was washed with water, and the product was extracted with ether. The combined organic layer was washed with brine, dried over anhydrous MgSO₄, and concentrated in vacuo. The crude product was purified by column chromatography to obtain the trans-lactones.

### 5. Alternative Procedure for the Preparation of Cis-Lactones [20].

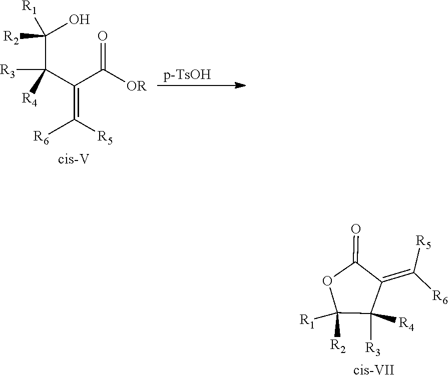

To a solution of cis-γ-hydroxyester (1.0 equiv) in DCM was added p-toluenesulfonic acid (0.1 equiv). The solution was stirred until judged as complete by TLC. After partitioning between water and dichloromethane, the organic layer was dried over Na₂SO₄. The crude product was purified by column chromatography to obtain the desired cis-lactone.

### 6. General Procedure for Aminolactone Via Conjugate Addition to VII (Compound Formula VI) [26].

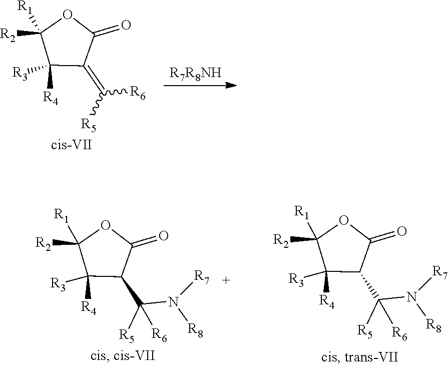

To a solution of cis-α-methylenelactone (1.0 equiv) in DCM was added N,N-dialkylamine (3.0 equiv) in one portion at room temperature. The solution was rapidly stirred until complete as judged by TLC analysis. The crude mixture was directly concentrated in vacuo to remove the solvent and excess amine. If deemed necessary, the crude reaction mixture was purified by flash column chromatography with amine additive to yield the desired aminolactone.

7. General Procedure for Aminolactonization. Direct Synthesis of Aminolactones (Compound Formula VI) from γ-Hydroxyesters (Compound Formula V) [27].

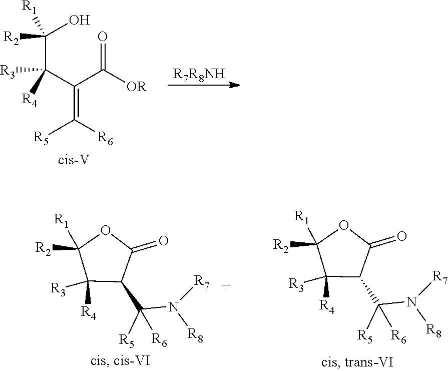

To a solution of cis-7-hydroxyester (1.0 equiv) in dichlormethane was added N,N-dialkylamine (3.0 equiv) in one portion at room temperature. The solution was rapidly stirred until complete as judged by TLC analysis. The crude mixture was directly concentrated in vacuo to remove the solvent and excess amine. If deemed necessary, the crude reaction mixture was purified by flash column chromatography with amine additive to yield the desired aminolactone.

One-pot synthesis and purification of AAMGBLs via fumarate salt formation.
(i) Barbier reaction of 4-(benzyloxy)-3-methoxybenzaldehyde (benzyl vanillin)

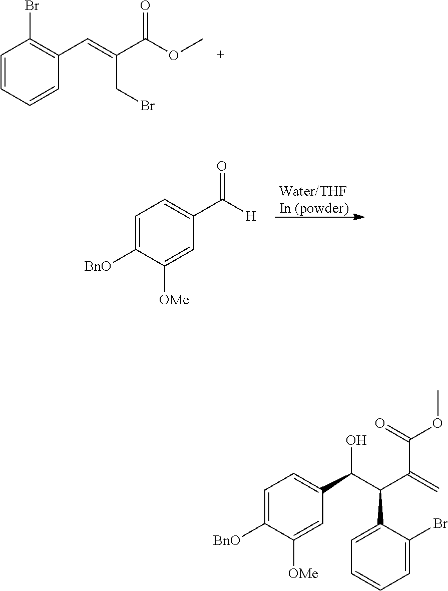 [SMILES: COC(=O)/C(=C/c1ccccc1Br)CBr ; COc1cc(C=O)ccc1OCc1ccccc1 ; C=C(C(=O)OC)[C@@H](c1ccccc1Br)[C@H](O)c1ccc(OCc2ccccc2)c(OC)c1]

Into a round bottom flask charged with a magnetic stirbar was added 1.0 equiv. allylic bromide. This material was suspended in deionized water to produce a 1.0 M suspension. 1.5 volumetric equiv. of THF (w/respect to H₂O) were then added with vigorous stirring, followed by 1.5 equiv. electrophile (aldehyde or ketone) and 1.3 equiv. indium powder. The reaction mixture was stirred for 2-12 hours (reaction followed by TLC) at room temperature, and then was partitioned between EtOAc and deionized water. After separating the organic layer, the aqueous layer was extracted with multiple portions (as necessary) of ethyl acetate. The combined organic layers were washed with brine, dried with Na₂SO₄, filtered, and concentrated.
(ii) Aminolactonization of hydroxy ester (Diastereoselective Synthesis of α-(Aminomethyl)-7-Butyrolactones via a Catalyst-free Aminolactonization. Ramachandran, P. V.; Nicponski, D. R. Chem. Commun. 2014, 50, 15216-15219).

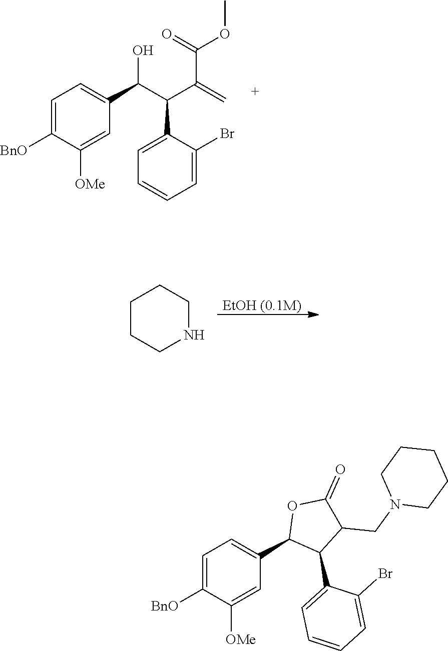 [SMILES: C=C(C(=O)OC)[C@@H](c1ccccc1Br)[C@H](O)c1ccc(OCc2ccccc2)c(OC)c1 ; C1CCNCC1 ; COc1cc([C@H]2OC(=O)C(CN3CCCCC3)[C@H]2c2ccccc2Br)ccc1OCc1ccccc1]

The concentrated crude reaction mixture from the previous Barbier reaction was dissolved in absolute ethanol to make a 0.1 M solution with respect to the original equivalents of allyl bromide used. To this solution 2.0 equiv. of piperidine were added, and the mixture was stirred at room temperature until the reaction was complete by TLC. The volatiles were evaporated from the reaction, and the mixture was dissolved in diethyl ether to make a 0.1 M solution with respect to the original allyl bromide.
(iii) Preparation of fumarate salt of aminolactone and recovery of aminolactone (Diastereoselective Synthesis of α-(Aminomethyl)-γ-Butyrolactones via a Catalyst-free Aminolactonization. Ramachandran, P. V.; Nicponski, D. R. Chem. Commun. 2014, 50, 15216-15219).

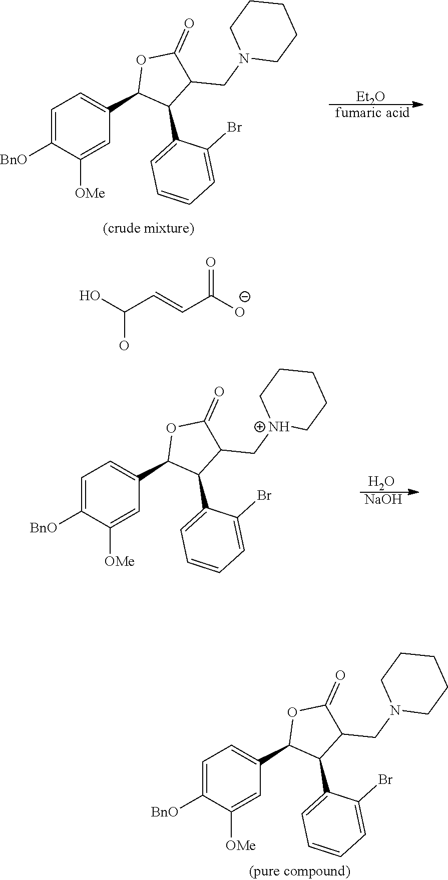

Fumaric acid (2 equiv) is added to the above solution, and the mixture is stirred vigorously for 30-60 minutes. The solution becomes cloudy and white over this time as the fumarate salt precipitated out of the solution. This suspension is then centrifuged and the supernatant decanted. The remaining solid is resuspended with additional diethyl ether, and the centrifuge, decant process is repeated 3 additional times. After the final decantation of the supernatant, the solid is dissolved in water. 3 M NaOH is added, dropwise, to the above solution until no more solid precipitates out of the aqueous solution. The solid is extracted from the aqueous mixture with 3 portions of diethyl ether, the organic extracts are combined, washed with brine, dried over sodium sulfate, filtered and concentrated in vacuo, when the pure aminolactone is obtained.

### Bio-Assay

Provided is a method for controlling Clostridioides difficile bacterial cell growth by arresting the cell cycle or causing cell death. The method involves treating a cell with a compound with the general structural formula I, II, or III, or an organic or inorganic salt, solvate, or hydrate thereof. A cell can be a cancerous cell or any other cell with abnormal growth. Although focus was on Clostridioides difficile bacterial cells, the efficacy of the lactones and aminolactones was also examined against other bacterial cells, such as Staphylococcus aureus, Enterococcus faecalis, Enterococcus faecium, Escherichia coli, and fungal cells, such as Candida albicans.

Method: The minimum inhibitory concentration (MIC) of the compounds and control drugs, linezolid (antibiotic), vancomycin (antibiotic), 5-fluorocytosine (5-FC, antifungal drug), and fluconazole (antifungal drug) was determined using the broth microdilution method according to the guidelines outlined by the Clinical and Laboratory Standards Institute (CLSI) [28, 29] against clinically relevant bacterial (methicillin-resistant Staphylococcus aureus (MRSA), vancomycin-resistant Enterococcus faecalis (VRE), vancomycin-resistant Enterococcus faecium (VRE), Clostridioides difficile and Escherichia coli) and fungal (Candida albicans) strains. A solution equivalent to 0.5 McFarland standard was prepared from each strain and diluted in cation-adjusted Mueller-Hinton broth (for S. aureus and E. coli), tryptone soy broth (for E. faecalis), or brain heart infusion broth (for E. faecium) to achieve a bacterial inoculum of about 5×10⁵ CFU/mL. C. difficile strains were cultured in brain heart infusion broth supplemented with hemin and vitamin K. C. albicans was diluted in RPMI medium supplemented with 3-(N-morpholino) propanesulfonic acid to achieve a fungal concentration of about 1.5×10³ CFU/mL. Compounds and control drugs were added in the first row of the 96-well plates and diluted with media containing bacteria (to achieve a concentration gradient ranging from 128 to 1 μg/mL). For MRSA, VRE, and E. coli strains, plates were then incubated aerobically at 37° C. for at least 18 hours. C. albicans was incubated aerobically at 37° C. for 24 hours. Plates containing C. difficile were incubated anaerobically at 370 C for 48 hours. The partial results of the MICs reported in Table 1 are the lowest concentration that the compound/drug completely inhibited the visual growth of bacteria/fungi.

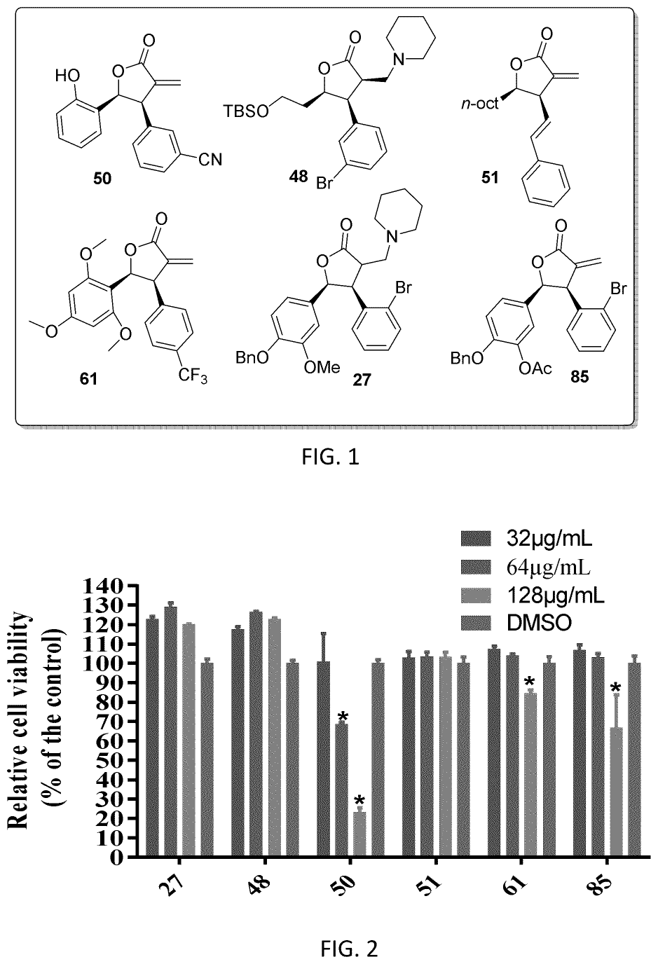

Results: The preliminary whole-cell screening assays established a discernible relationship between the substitution pattern and the anti-bacterial activity for a series of racemic cis-β,γ-disubstituted-α-methylene- and -aminomethyl-γ-butyrolactones. The assays revealed that (i) more than half of the 120 lactones were inactive against all the bacterial strains assayed, (ii) few of the molecules were active against all of the gram-positive bacterial cells with different degrees of bacteriostatic capability, and six of the molecules (27, 48, 50, 51, 61 and 85, see FIG. 1 for detailed structure) displayed highly selective antibacterial activity against C. difficile (Table 1). Among these, the piperidinomethyl lactone 27 showed highly potent activity against various pathogenic strains of C. difficile, similar to vancomycin.

TABLE 1
The minimum inhibitory concentration (MIC in μg/mL) of
lactones and control drugs screened against bacterial strains.

| | | | | E. coli |
|---|---|---|---|---|
| Lactone/ | MRSA | E. faecalis | Clostridioides | (TolC |
| Control drugs | (USA300)^{a} | (VRE)^{b} | difficile | mutant) |
| 27 | >128 | >128 | 1 | >128 |
| 48 | >128 | >128 | 8 | >128 |
| 50 | >128 | >128 | 8 | >128 |
| 51 | >128 | >128 | 8 | >128 |
| 61 | >128 | >128 | 8 | >128 |
| 85 | >128 | >128 | 4 | >128 |
| Linezolid | 1 | 1 | not | 16 |
| | | | tested | |
| Vancomycin | 1 | 32 | 1 | not |
| | | | | tested |
| Metronidazole | not | not | 0.25 | not |
| | tested | tested | | tested |

Assay Against Clinical Strains of C. difficile

Since lactones 27, 48, 50, 51, 61 and 85 did not inhibit the tested strains of MRSA and E. faecium, they were further tested against clinical strains of C. difficile to assess their selective activity against C. difficile. Given these compounds were inactive against MRSA, VRE and C. albicans and exhibited moderate antibacterial activity against C. difficile BAA-1870, their activity against additional strains of C. difficile was examined.

The antibacterial activity of compounds 48, 50, 51, 61 and 85 against a panel of Clostridioides difficile strains was evaluated. A bacterial solution equivalent to 0.5 McFarland standard was prepared and diluted in brain heart infusion broth supplemented with hemin and vitamin K. Compounds and control drugs were added in the first row of 96-well plates and serially diluted with media containing bacteria (to achieve a concentration gradient ranging from 128 to 1 μg/mL). Plates were then incubated anaerobically at 370 C for 48 hours. MICs reported in Tables 2 and 3 are the minimum concentration of the compounds and control drugs that completely inhibited the visual growth of bacteria.

Screening of the Lactones Against Hypervirulent C. difficile Strains:

### Screening of the Lactones Against Normal Human Gut Microflora (Microbiota):

The activity of the selected lactones against the human gut microbiota was also examined. Microbiota interfere with C. difficile's ability to colonize the gastrointestinal tract and subsequently cause infection. Many antibiotics inhibit/kill the endogenous microflora, which permits enhanced colonization by C. difficile and infection. Three different bacterial species that are present in the gut microflora were used in this experiment; Bifidobacterium (anaerobic Gram-positive bacteria), Bacteroides (anaerobic Gram-negative bacteria) and Lactobacillus (microaerophilic Gram-positive bacteria). Ideal compounds/drugs are those with selective antibacterial activity against C. difficile so as to protect the indigenous microflora present in the GI tract from being damaged/killed (which will exacerbate infections caused by C. difficile).

TABLE 2
The minimum inhibitory concentration (MIC in μg/mL)
of compounds and control drugs against hypervirulent
Clostridioides difficile strains.

| | Lactones/Control Drugs | | | | | | | | |
|---|---|---|---|---|---|---|---|---|---|
| CD | MIC μg/mL | | | | | | | | |
| Strains | 27 | 48 | 50 | 51 | 61 | 85 | Vanc | Met | Fid |
| 81 | 2 | 16 | >128 | 16 | 16 | 4 | 0.25 | 0.125 | 0.03 |
| 82 | 2 | 8 | 64 | 16 | 16 | 2 | 0.5 | 0.25 | 0.3 |
| 83 | 2 | 8 | 128 | 16 | 64 | 4 | 0.5 | 0.25 | 0.015 |
| 84 | 4 | 8 | >128 | 64 | 64 | 4 | 0.25 | 0.25 | 0/03 |
| 85 | 2 | 8 | >128 | 64 | 64 | 2 | 2 | 0.25 | 0.03 |
| 86 | 2 | 8 | 128 | 32 | 32 | 4 | 0.5 | 0.125 | 0.06 |
| 87 | 2 | 4 | 128 | 32 | 8 | 2 | 0.5 | 0.125 | 0.015 |
| 90 | 1 | 4 | 128 | 4 | 16 | 2 | 1 | 0.125 | ≤0.007 |
| 49279 | 2 | 8 | >128 | 16 | 64 | 4 | 1 | 0.125 | ≤0.007 |
| 49287 | 1 | 4 | 128 | 8 | 8 | 2 | 0.25 | ≤0.06 | ≤0.007 |
| 49291 | 1 | 4 | 64 | 8 | 8 | 2 | 0.25 | 0.125 | ≤0.007 |

TABLE 3
The minimum inhibitory concentration (MIC in μg/mL)
of compounds and control drugs against clinically important
Clostridioides difficile strains.

| CD | Lactones/Control Drugs MIC μg/mL | | | | | | | | |
|---|---|---|---|---|---|---|---|---|---|
| Strains | 27 | 48 | 50 | 51 | 61 | 85 | Vanc | Met | Fid |
| P6 | 2 | 8 | 128 | 8 | 8 | 4 | 1 | ≤0.06 | ≤0.007 |
| P7 | 1 | 2 | 128 | 16 | 8 | 4 | 1 | 0.25 | ≤0.007 |
| P19 | 2 | 2 | 32 | 16 | 8 | 2 | 1 | 0.25 | 0.03 |
| P13 | 1 | 2 | 32 | 2 | 8 | 4 | 1 | ≤0.06 | ≤0.007 |
| 16 | 1 | 4 | 128 | 16 | 16 | 4 | 1 | 0.25 | 0.015 |
| 18 | 2 | 2 | 32 | 2 | 2 | 4 | 0.5 | ≤0.06 | ≤0.007 |
| 19 | 1 | 4 | 128 | 16 | 8 | 4 | 1 | 0.125 | 0.015 |
| 43255 | 1 | 8 | 16 | 16 | 4 | 4 | 1 | 0.125 | 0.03 |
| 1801 | 1 | 8 | 128 | 32 | 16 | 2 | 1 | 0.125 | 0.015 |

Vanc = vancomycin,
Met = Metronidazole,
Fid = Fidaxomycin

TABLE 4
The minimum inhibitory concentration (MIC in μg/mL) of compounds
and control drugs against human normal gut microbiota.

| Microbiota | Lactones/Control Drugs MIC μg/mL | | | | | | | | |
|---|---|---|---|---|---|---|---|---|---|
| strains | 27 | 48 | 50 | 51 | 61 | 85 | Vanc | Met | Fid |
| L. gasseri | >128 | >128 | >128 | >128 | >128 | >128 | >128 | >128 | 1 |
| HM 407 | | | | | | | | | |
| L. casei | >128 | >128 | >128 | >128 | >128 | >128 | >128 | >128 | 2 |
| HM 334 | | | | | | | | | |
| L. reuteri | >128 | >128 | >128 | >128 | >128 | >128 | >128 | >128 | 128 |
| HM 120 | | | | | | | | | |
| L. crispatus | >128 | >128 | >128 | >128 | >128 | >128 | ≤1 | >128 | 2 |
| HM 421 | | | | | | | | | |
| L. crispatus | >128 | >128 | >128 | >128 | >128 | >128 | ≤1 | >128 | ≤1 |
| HM 375 | | | | | | | | | |
| Ba.* | >128 | >128 | >128 | >128 | >128 | >128 | 64 | ≤1 | >128 |
| fragilis | | | | | | | | | |
| HM 709 | | | | | | | | | |
| Ba.* | >128 | >128 | >128 | >128 | >128 | >128 | 16 | ≤1 | >128 |
| fragilis | | | | | | | | | |
| HM 710 | | | | | | | | | |
| Ba.* | >128 | >128 | >128 | >128 | >128 | >128 | 16 | ≤1 | >128 |
| fragilis | | | | | | | | | |
| HM 711 | | | | | | | | | |
| Ba.* | >128 | >128 | >128 | >128 | >128 | >128 | 16 | ≤1 | >128 |
| fragilis | | | | | | | | | |
| HM 714 | | | | | | | | | |
| Ba.*dorsei | >128 | >128 | >128 | >128 | >128 | >128 | 128 | ≤1 | >128 |
| HM 29 | | | | | | | | | |
| Ba.*dorsei | >128 | >128 | >128 | >128 | >128 | >128 | 8 | ≤1 | >128 |
| HM 717 | | | | | | | | | |
| Bi.** breve | >128 | >128 | >128 | >128 | >128 | >128 | ≤1 | ≤1 | >128 |
| HM 856 | | | | | | | | | |
| Bi.** | >128 | >128 | >128 | >128 | >128 | >128 | 64 | 64 | 32 |
| longum | | | | | | | | | |
| HM 848 | | | | | | | | | |

*Ba = Bacteroides,
**Bi = Bifidobacterium.
Vanc = vancomycin,
Met = metronidazole,
Fid = fidaxomicin

Bifidobacterium and Bacteroides were first grown for 48 hours at 37° C., anaerobically, using brain heart infusion supplemented (BHIS) agar (brain heart infusion agar supplemented with hemin, vitamin K and L-cysteine). Lactobacillus was cultivated on MRS agar and incubated in the presence of 5% CO₂ for 48 hours at 37° C. Each bacterium was suspended in phosphate-buffered saline (PBS) to achieve 0.5 McFarland standard and subsequently diluted in brain heart infusion supplemented broth (for Bifidobacterium and Bacteroides) or in MRS broth (for Lactobacillus) to achieve a bacterial inoculum of approximately 5×10⁵ CFU/mL. Bacteria were then seeded in 96-well plates containing serial dilutions of the compounds and incubated for 48 hours. MICs reported in Table 4 are the minimum concentration of compound/control drug that inhibited the visual growth of bacteria.

It is extremely noteworthy that none of the selected lactones tested inhibit any bacteria of the normal gut microflora, which is extremely important and advantageous to develop them as potential drugs to be used against C. difficile. In contrast, all three control drugs tested had a deleterious effect on certain bacterial species (for example, metronidazole inhibited growth of species of Bacteroides and Bifidobacterium).

### In Vivo Safety Profile and Toxicity

Aminomethyllactone 27 was evaluated for any possible toxicity at oral doses higher than the anticipated dose that will be used for antimicrobial efficacy. The maximum tolerated dose (MTD) in mice was calculated and was expected to be several folds higher than therapeutic dose. The MTD is a dose that produces neither mortality nor more than a 10% decrement in body weight nor clinical signs of toxicity or a significant change in renal and/or hepatic function in the survivors. Having shown that 10 mg/kg in mice is not toxic in our initial mouse model studies, 10, 25, 50, 100, 200, 400 mg/kg doses of 27 were chosen, each dose was given one time to one mouse, and the mice were observed daily for 14 days for body weight gain, clinical signs of abnormality, and renal and hepatic function.

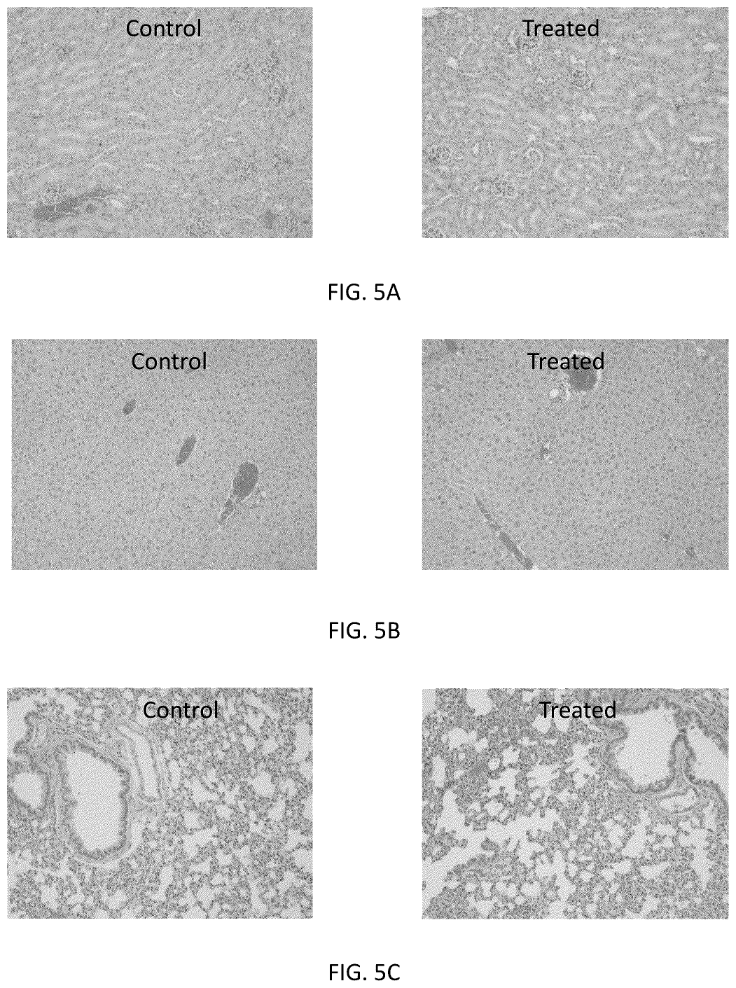

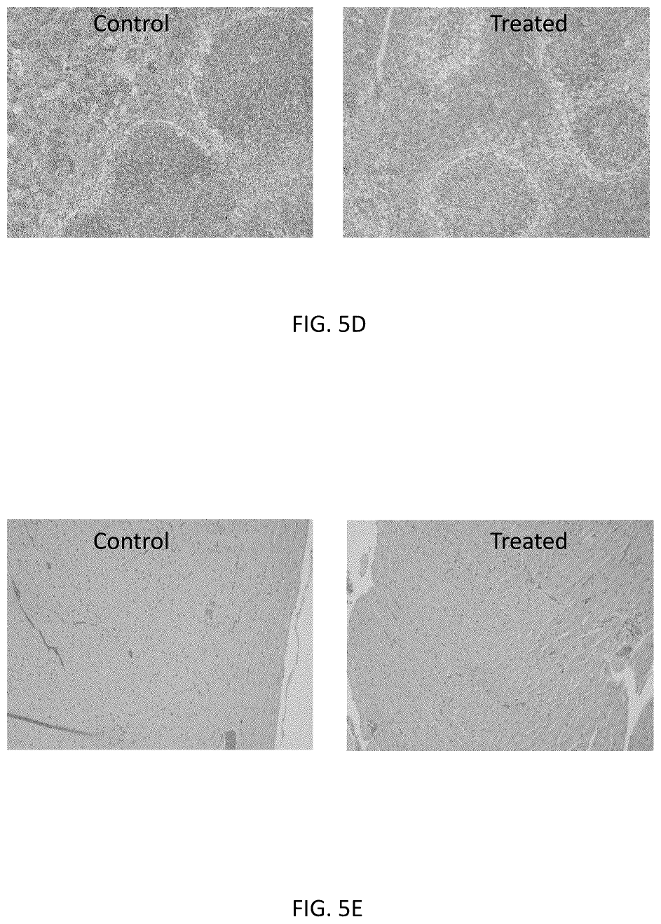

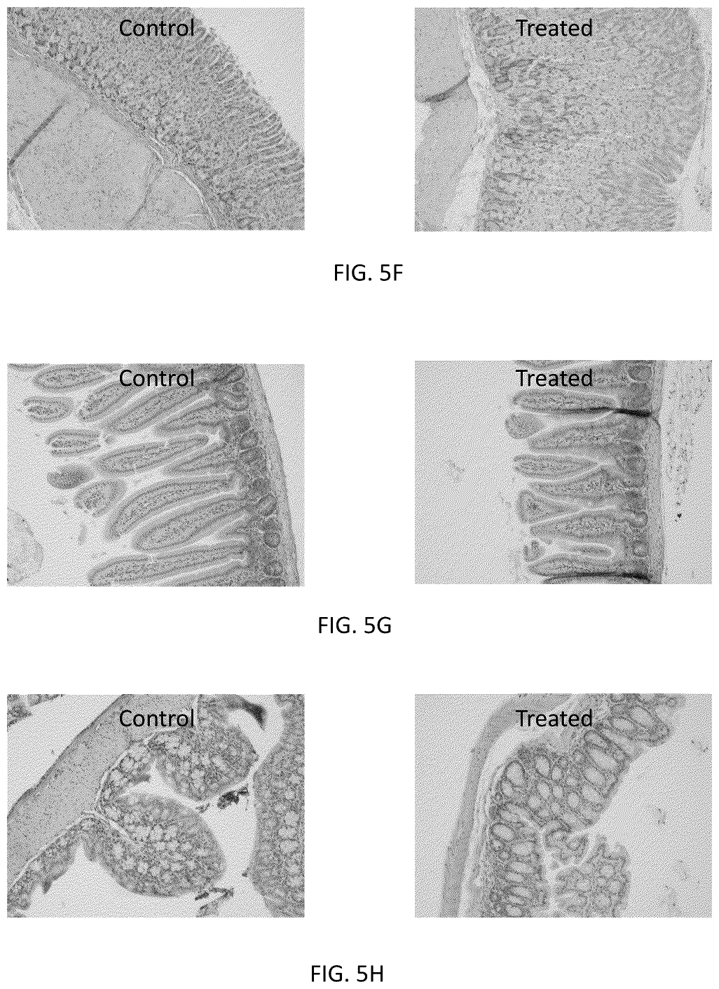

At the end of the study, all animals appeared to be healthy and have good body weight. They were then euthanized, and the major organ (heart, liver, lung, kidney, spleen, and intestines) tissues were harvested for histological examination of toxicity. The evaluator was blinded to the treatment status of each mouse at the time of examination. Overall, the tissues examined appeared to be within normal histologic limits. The images from histology are shown in FIG. 5. Overall, 27 appears to be non-toxic to mice.

Antibiotic-Induced C. difficile-Associated Diarrhea Mouse Model

Preliminary in vivo experiments were carried out in two mouse models of antibiotic-induced C. difficile-associated diarrhea and two recurrence models to ensure the feasibility of using this new class of antibiotic orally against C. difficile infection.

### EXPERIMENTAL

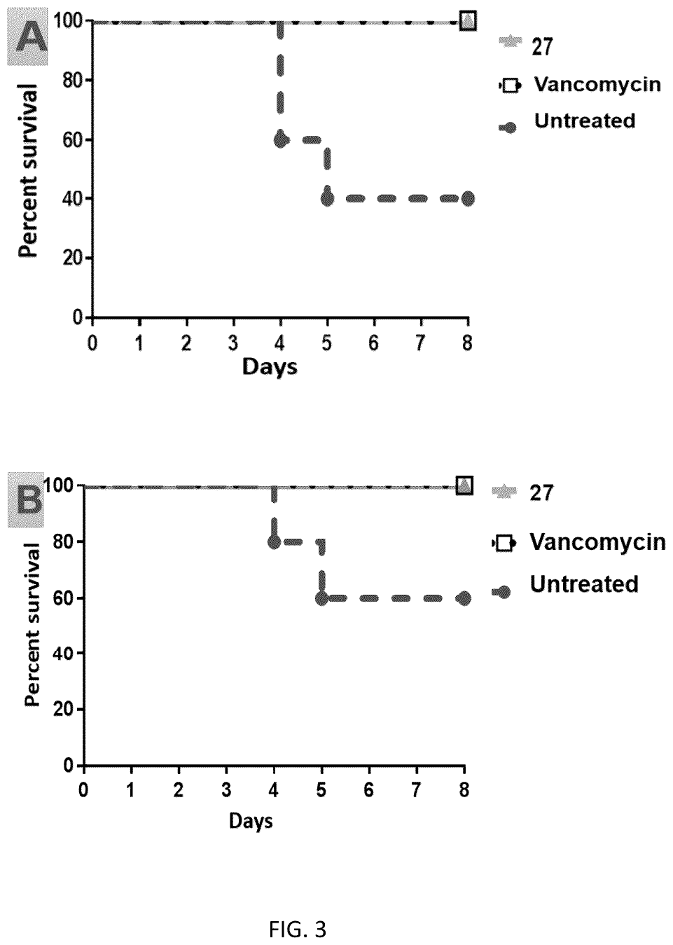

Groups of 12-weeks-old female C₅₇BL/6 mice were sensitized to CDI with two different protocols [32,33]. A—Mice were given antibiotic cocktail containing gentamicin (0.035 mg/mL), kanamycin (0.4 mg/ml), metronidazole (0.215 mg/mL), colistin (850 U/mL), and vancomycin (0.045 mg/mL) in drinking water for 5 days. Then mice were treated with clindamycin (10 mg/kg) two days after withdrawal of the antibiotic cocktail. One day after clindamycin treatment, mice were infected orally with 1.9×10⁶ CFU spore suspension of C. difficile ATCC 43255 [32]. Two hours after infection, animals were divided into groups (n=10), and groups received either vehicle (sterile water), Compound 27 (10 mg/kg), or vancomycin (10 mg/kg) by gavage daily for 5 days. B— In the second protocol mice were sensitized to CDI with cefoperazone (1.5 mg/mL) in the drinking water for 10 days. Afterwards, mice were switched to regular water for 2 days, followed by injecting clindamycin (10 mg/kg) intraperitoneally [38]. One day after clindamycin treatment, mice were infected and treated as group A above. As presented in FIGS. 3 A and B, aminomethyllactone 27 showed 100% protection at 10 mg/kg in the two mouse models used, same as vancomycin.

Sustained Treatment with 27 and Comparison with Vancomycin

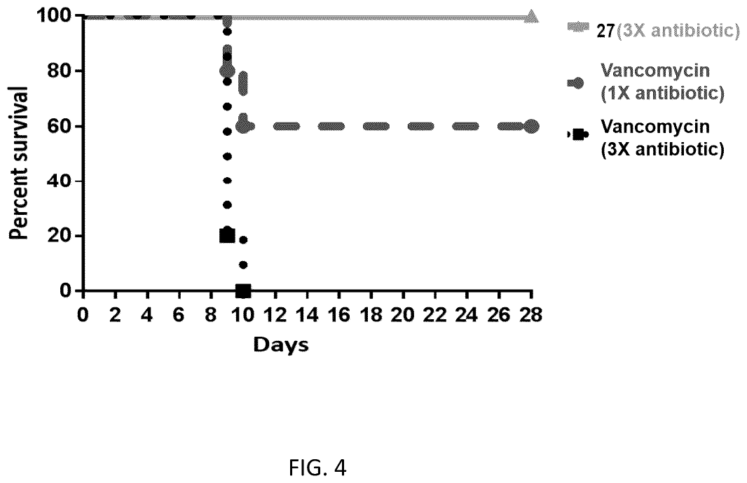

For mouse recurrence, two models were used for testing the efficacy of aminomethyllactone 27 for preventing recurrence after treatment with vancomycin. The two models used were A—Chen et al model [32] and B— a more aggressive protocol that was developed using modified Chen et al. protocol antibiotic cocktail (applying 3× times the doses of the antibiotics). In brief, groups of 12-weeks-old female C57BL/6 mice were sensitized to CDI by giving either the antibiotic cocktail described above or 3× the dose of antibiotics cocktail in the drinking water for 5 days. Then mice were treated with clindamycin (10 mg/kg) two days after withdrawal of the antibiotic cocktail. One day after clindamycin treatment, mice were infected orally with 1.9×10⁶ CFU spore suspension of C. difficile ATCC 43255 [32]. Two hours after infection, animals were divided into groups (n=10), and groups received either Compound 27 (10 mg/kg) or vancomycin (10 mg/kg) by gavage daily for 5 days. Treatment was stopped at day 5, and mice were monitored for an additional 23 days. Mice treated with compound 27 or vancomycin showed no signs of disease during therapy. However, from day 9 onward (4 days after vancomycin was discontinued), vancomycin-treated mice developed CDI. By day 11, only 60% of the mice treated with vancomycin had survived in the original protocol (lx antibiotic), and there was 0% survival in the aggressive antibiotic protocol (3× antibiotic). Oral treatment with identical doses of compound 27 resulted in 100% survival of mice with no recurrence for 28 days (FIG. 4). Symptomatic recurrence of CDI occurs in approximately 20% of patients and is challenging to treat [34-38]. In addition to subsequent prolongation of C. difficile shedding and transmission, 1 out of every 5 patients with CDI, who experienced a recurrence of the infection, died within 30 days of diagnosis [12]. Compound 27, which reduces recurrences, can potentially prolong the clinical utility of current antibiotics (vancomycin and fidaxomicin).

### In Vitro Cytotoxicity Analysis of the Lactones Against Caco-2 Cells

The in vitro toxicity studies were designed to evaluate potential toxicity of the synthesized alpha-methylene gamma butyrolactones (AMGBLs) and alpha aminomethyl gamma butyrolactones (AAGBLs) to provide crucial information about nontoxic doses to be used for future animal studies. Caco-2 cells, an immortalized cell line of human colorectal adenocarcinoma, primarily used as a model of the intestinal epithelial barrier, were chosen due to their presence in gastroinstestinal tract (GIT) [30,31].

Method: Compounds were assayed (at concentrations of 32, 64 and 128 μg/mL) against a human colorectal (Caco-2) cell line to determine the potential toxic effect to mammalian cells in vitro. Briefly, cells were cultured in Dulbecco's Modified Eagle Medium (DMEM) supplemented with 20% fetal bovine serum (FBS), non-essential amino acids (1×), and penicillin-streptomycin at 37° C. with CO₂ (5%). Control cells received DMSO alone at a concentration equal to that in drug-treated cell samples. The cells were incubated with compounds (in triplicate) in a 96-well plate at 37° C. with CO₂ (5%) for two hours. The assay reagent MTS 3-(4,5-dimethylthiazol-2-yl)-5-(3-carboxymethoxyphenyl)-2-(4-sulfophenyl)-2H-tetrazolium) (Promega, Madison, WI, USA) was subsequently added, and the plate was incubated for four hours. Absorbance readings (at OD490) were taken using a kinetic microplate reader (Molecular Devices, Sunnyvale, CA, USA). The quantity of viable cells after treatment with each compound was expressed as a percentage of the viability of DMSO-treated control cells (average of triplicate wells±standard deviation). The toxicity data were analyzed via a two-way ANOVA, with post hoc Dunnet's multiple comparisons test (P<0.05), utilizing GraphPad Prism 6.0 (GraphPad Software, La Jolla, CA) (FIG. 2).

FIG. 2 shows toxicity analysis of compounds 27, 48, 50, 51, 61, and 85 against human colorectal cells (Caco-2). Percent viable mammalian cells was measured as average absorbance ratio (test agent relative to DMSO) for cytotoxicity analysis of the compounds (tested in triplicate) at 32, 64 and 128 μg/mL against Caco-2 cells using the MTS 3-(4,5-dimethylthiazol-2-yl)-5-(3-carboxymethoxyphenyl)-2-(4-sulfophenyl)-2H-tetrazolium) Dimethyl sulfoxide (DMSO) was used as a negative control to determine a baseline measurement for the cytotoxic impact of each compound. The absorbance values represent an average of a minimum of three samples analyzed for each compound. Error bars represent standard deviation values for the absorbance values. A two-way ANOVA, with post hoc Dunnet's multiple comparisons test, determined a statistical difference (denoted by the asterisk, *) (P<0.05) between the values obtained for each compound and DMSO (negative control, used as solvent for the compounds).

Results: Compounds 27, 48 and 51 were non-toxic to Caco-2 cells up to a concentration of 128 μg/mL. Compounds 61 and 85 were found to be toxic to the Caco-2 cells at 128 μg/mL. Compound 50 was non-toxic to Caco-2 cells up to a concentration of 32 μg/mL.

Those skilled in the art will recognize that numerous modifications can be made to the specific implementations described above. The implementations should not be limited to the particular limitations described. Other implementations may be possible.

It is intended that that the scope of the present methods and compositions be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

### REFERENCES

1. Yassin S F, Young-Fadok T M, Zein N N, Pardi D S. Clostridium difficile-associated diarrhea and colitis. Mayo Clin. Proc. 2001. 76 (7): p. 725-730.
2. Bartlett J G. Antibiotic-associated diarrhea. Clin. Infect. Dis. 1992: 15: 573-581.
3. Wiström J, Norrby S R, Myhre E B, Eriksson S, Granström G, Lagergren L, Englund G, Nord C E, Svenungsson B. 2001. J. Antimicrob. Chemother. 2001. 47:43-50.
4. Abt, M. C., P. T. McKenney, and E. G. Pamer, Clostridium difficile colitis: pathogenesis and host defence. Nat Rev Microbiol, 2016. 14(10): p. 609-20.
5. Venugopal, A. A. and S. Johnson, Fidaxomicin: a novel macrocyclic antibiotic approved for treatment of Clostridium difficile infection. Clin Infect Dis, 2012. 54(4): p. 568-74.
6. Crawford, T., E. Huesgen, and L. Danziger, Fidaxomicin: Am J Health Syst Pharm, 2012. 69(11): p. 933-43.
7. Hardesty, J. S. and P. Juang, Fidaxomicin: a macrocyclic antibiotic for the treatment of Clostridium difficile infection. Pharmacotherapy, 2011. 31(9): p. 877-86.
8. Louie, T. J., et al., Fidaxomicin versus vancomycin for Clostridium difficile infection. N Engl J Med, 2011. 364(5): p. 422-31.
9. Mullane, K. M., et al., Efficacy of fidaxomicin versus vancomycin as therapy for Clostridium difficile infection in individuals taking concomitant antibiotics for other concurrent infections. Clin Infect Dis, 2011. 53(5): p. 440-7.
10. Comely, O. A., et al., Treatment of first recurrence of Clostridium difficile infection: fidaxomicin versus vancomycin. Clin Infect Dis, 2012. 55 Suppl 2: p. S154-61.
11. Locher, H. H., et al., In vitro and in vivo antibacterial evaluation of cadazolid, a new antibiotic for treatment of Clostridium difficile infections. Antimicrob Agents Chemother, 2014. 58(2): p. 892-900.
12. CDC, Antibiotic Resistance Threats in the United States, 2013. 2013, Centers for Disease Control and Prevention. p. 1-114.
13. Ramachandran, P. V.; Yip-Schneider, M. T.; Schmidt, C. M. Natural and Synthetic □,□-Unsaturated Carbonyls for NF-□B Inhibition: A Mini-review Future Med. Chem. 2009, 1, 179.
14. (a) Kitson, R. R. A.; Millemaggi, A.; Taylor, R. J. K. Angew. Chem. Int. Ed. 2009, 48, 9426. (b) Kupchan, S. M.; Fessler, D. C.; Eakin, M. A.; Ciacobbe, T. J. Science 1970, 168, 376.
15. (a) Guzman M L, Rossi R M, Karnischky L, Li X, Peterson D R, et al. Blood 2005 105: 4163-4169. (b) Steele A J, Jones D T, Ganeshaguru K, Duke V M, Yogashangary B C, et al. (2006) Leukemia 20: 1073-1079.
16. Neelakantan, S.; Nasim, S.; Guzman, M. I.; Jordan, C. T.; Crooks, P. A. Aminoparthnolides as novel anti-leukemic agents: Discovery of the NF-κB inhibitor, DMAPT (LC-1). Bioorg. Med. Chem. Lett. 2009, 19, 4346-4349.
17. Nasim, S.; Crooks, P. A. Antileukemic activity of aminoparthnolide analogs. Bioorg. Med. Chem. Lett. 2008, 18, 3870-3873.
18. Ramachandran P V, Pratihar D, Nair H N, Walters M, Smith S, Yip-Schneider M T, Wu H, Schmidt C M. Bioorg. Med. Chem. Lett. 2010, 20, 6620-23.
19. Blakeman, J. P., Atkinson, P., Antimicrobial properties and possible role in host-pathogen interactions of parthenolide, a sesquiterpene lactone isolated from glands of Chrysanthemum parfhenium. Physiological Plant Pathology, 1979, 15, 183-192.
20. Park, B. K., Nakagawa, M., Hirota, A., Nakayama, M. Methylenolactocin, a novel antitumor antibiotic from penicillium sp. J. Antibiotics. 1988, 41 (6): 751-758.
21. Kim, K. H.; Lee, H. S.; Kim, S. H.; Lee, K. Y.; Lee, J-E; Kim, J. N. Lactonization, Isomerization, and Aerobic Oxidation of α-Methylene-γ-hydroxyester; Bull. Korean. Chem. Soc. 2009, 30, 1012.
22. Ishiyama, T.; Ahiko, T-A.; Miyaura, N. Acceleration Effect of Lewis Acid in Allylboration of Aldehydes: Catalytic, Regiospecific, Diastereospecific, and Enantioselective Synthesis of Homoallyl Alcohols. J. Am. Chem. Soc. 2002, 124, 12414.
23. Ramachandran, P. V.; Garner, G.; Pratihar, D. Synthesis of (E)- and (Z)-α-Alkylidene-γ-aryl-γ-butyrolactones via Alkenylalumination of Oxiranes Org. Lett. 2007, 9, 4753.
24. Park, B. R.; Kim, K. H.; Kim, J. N. Tetrahedron Lett. 2010, 51, 6568.
25. Kennedy, J. W. J.; Hall, D. G. Dramatic Rate Enhancement with Preservation of Stereospecificity in the First Metal-Catalyzed Additions of Allylboronates. J. Am. Chem. Soc. 2002, 124, 11586.
26. Ramachandran, P. V.; Nicponski, D. R. Nair, H. N. G.; Gagare, P. D.; Helppi, M.; Schmidt, C. M.; Yip-Schneider, M. T., Synthetic α-(aminomethyl)-γ-butyrolactones and their anti-pancreatic cancer activities. Bioorg. Med. Chem. Lett. 2013, 23, 6911-6914
27. Ramachandran, P. V.; Nicponski, D. R., Chem. Commun. 2014, 50, 15216-15219.
28. CLSI, Clinical and Laboratory Standards Institute. M7-A9 Methods for dilution antimicrobial susceptibility tests for bacteria that grow aerobically—Ninth Edition, Wayne, P A, 2012.
29. CLSI, Clinical and Laboratory Standards Institute. M27-A3 Reference Method for Broth Dilution Antifungal Susceptibility Testing of Yeasts; Third Edition, Wayne, P A, 2008.
30. Mohamed, M. F., et al., Antibacterial Activity of Novel Cationic Peptides against Clinical Isolates of Multi-Drug Resistant Staphylococcus pseudintermedius from Infected Dogs. Plos One, 2014. 9(12).
31. Mohamed, M. F., A. Abdelkhalek, and M. N. Seleem, Evaluation of short synthetic antimicrobial peptides for treatment of drug-resistant and intracellular Staphylococcus aureus. Sci Rep, 2016. 6: p. 29707.
32. Chen, X., et al., A mouse model of Clostridium difficile-associated disease. Gastroenterology, 2008. 135(6): p. 1984-92.
33. Theriot, C. M., et al., Cefoperazone-treated mice as an experimental platform to assess differential virulence of Clostridium difficile strains. Gut Microbes, 2011. 2(6): p. 326-34.
34. Eyre, D. W., et al., Predictors of first recurrence of Clostridium difficile infection: implications for initial management. Clin Infect Dis, 2012. 55 Suppl 2: p. S77-87.
35. Comely, O. A., et al., Treatment of first recurrence of Clostridium difficile infection: fidaxomicin versus vancomycin. Clin Infect Dis, 2012. 55 Suppl 2: p. S154-61.
36. Petrella, L. A., et al., Decreased cure and increased recurrence rates for Clostridium difficile infection caused by the epidemic C. difficile BI strain. Clin Infect Dis, 2012. 55(3): p. 351-7.
37. Jung, K. S., et al., Risk Factors for Treatment Failure and Recurrence after Metronidazole Treatmentfor Clostridium difficile-associated Diarrhea. Gut Liver, 2010. 4(3): p. 332-7.
38. Kelsen, J. R., et al., Recurrence rate of Clostridium difficile infection in hospitalized pediatric patients with inflammatory bowel disease. Inflamm Bowel Dis, 2011. 17(1): p. 50-5.

### NUMBERED EMBODIMENTS

Embodiment 1 relates to compound having the formula

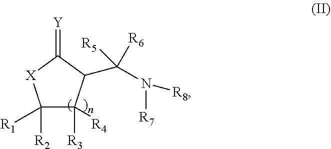

or a pharmaceutically acceptable salt thereof,
wherein
X is O, NH, NR, or S;
Y is O, NH, NR, or S;
n is 1, 2, 3, or 4;
R is H or alkyl;
R₁, R₂, R₃, R₄, R₅, and R₆ are independently hydrogen, halogen, hydroxyl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
R₇ and R₈ are independently hydrogen, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; or R₇ and R₈ are taken together with the N to which they are attached to form a ring system.

Embodiment 2 relates to a compound of Embodiment 1, wherein the compound is of formula

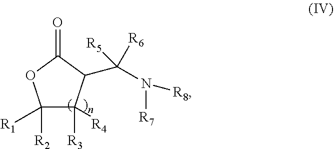

or a pharmaceutically acceptable salt thereof,
wherein
n is 1, 2, 3, or 4;
R₁, R₂, R₃, R₄, R₅, and R₆ are independently hydrogen, halogen, hydroxyl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
R₇ and R₈ are independently hydrogen, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; or R₇ and R₈ are taken together with the N to which they are attached to form a ring system.

Embodiment 3 relates to a compound of Embodiment 1, wherein the compound is of formula

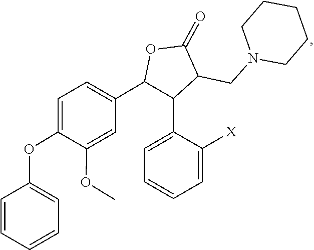

or a pharmaceutically acceptable salt thereof, wherein X is a halo.

Embodiment 4 relates to a compound of Embodiment 1, wherein the compound is of formula

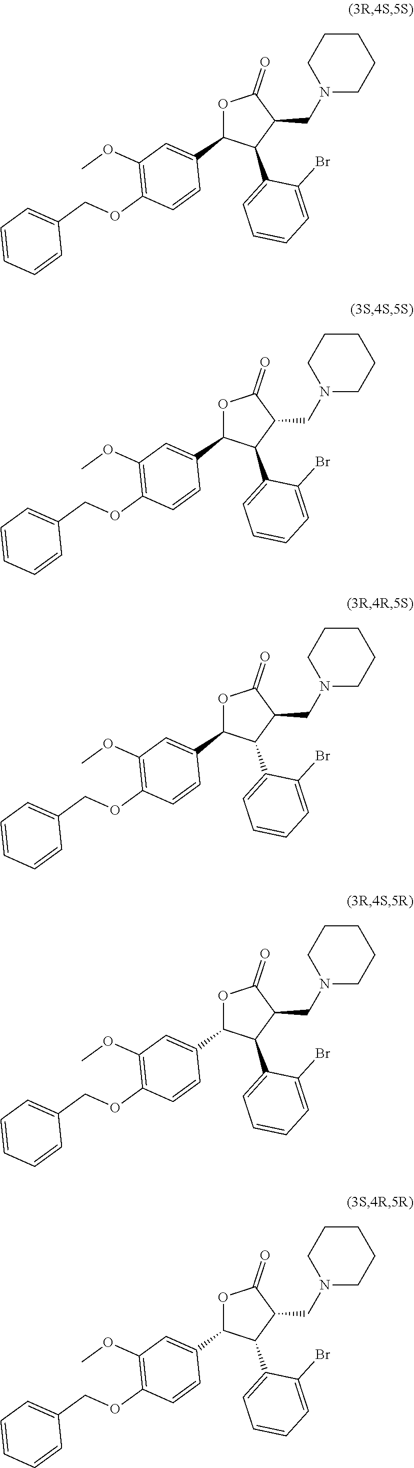 [SMILES: COc1cc([C@H]2OC(=O)[C@@H](CN3CCCCC3)[C@H]2c2ccccc2Br)ccc1OCc1ccccc1 ; COc1cc([C@H]2OC(=O)[C@H](CN3CCCCC3)[C@H]2c2ccccc2Br)ccc1OCc1ccccc1 ; COc1cc([C@H]2OC(=O)[C@@H](CN3CCCCC3)[C@@H]2c2ccccc2Br)ccc1OCc1ccccc1 ; COc1cc([C@@H]2OC(=O)[C@@H](CN3CCCCC3)[C@H]2c2ccccc2Br)ccc1OCc1ccccc1 ; COc1cc([C@@H]2OC(=O)[C@H](CN3CCCCC3)[C@@H]2c2ccccc2Br)ccc1OCc1ccccc1]

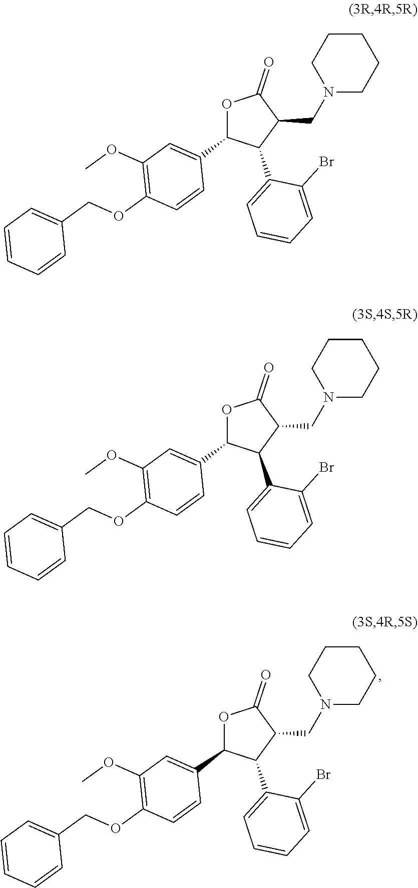 [SMILES: COc1cc([C@@H]2OC(=O)[C@@H](CN3CCCCC3)[C@@H]2c2ccccc2Br)ccc1OCc1ccccc1 ; COc1cc([C@@H]2OC(=O)[C@H](CN3CCCCC3)[C@H]2c2ccccc2Br)ccc1OCc1ccccc1 ; COc1cc([C@H]2OC(=O)[C@H](CN3CCCCC3)[C@@H]2c2ccccc2Br)ccc1OCc1ccccc1]

or a pharmaceutically acceptable salt thereof.

Embodiment 5 relates to a compound of formula

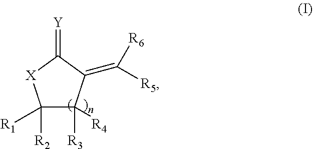

wherein
X is O, NH, NR, or S;
Y is O, NH, NR, or S;
n is 1, 2, 3, or 4; and
R₁, R₂, R₃, R₄, R₅, and R₆ are independently hydrogen, halogen, hydroxyl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted.

Embodiment 6 relates to a compound of Embodiment 5, wherein the compound is of formula

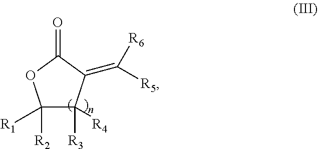

or a pharmaceutically acceptable salt thereof, wherein
n is 1, 2, 3, or 4; and
R₁, R₂, R₃, R₄, R₅, and R₆ are independently hydrogen, halogen, hydroxyl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl.

Embodiment 7 relate to a pharmaceutical composition comprising one or more compounds of any of Embodiments 1-6, together with one or more pharmaceutically acceptable diluents, excipients or carriers.

Embodiment 8 relates to the pharmaceutical composition of Embodiment 7, which is nanoparticulate.

Embodiment 9 relates to the pharmaceutical composition of Embodiment 7 or 8, further comprising one or more other antibiotics.

Embodiment 10 relates to a method for treating a patient with an infection comprising administering a therapeutically effective amount of one or more compounds of Embodiments 1-6, or a pharmaceutically acceptable salt thereof, or a pharmaceutical composition of any one of Embodiments 7-9 to a patient in need thereof.

Embodiment 11 relates to the method of Embodiment 10, wherein said infection is caused by Clostridioides difficile.

Embodiment 12 relates to the use of a compound or a pharmaceutically acceptable salt thereof according to any of claims 1-6 in the manufacture of a medicament for treating Clostridioides difficile infection in a subject.

1. A compound having the formula 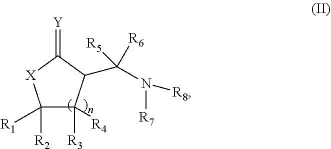
or a pharmaceutically acceptable salt thereof,
wherein
X is O, NH, NR, or S;
Y is O, NH, NR, or S;
n is 1, 2, 3, or 4;
R is H or alkyl;
R₁, R₂, R₃, R₄, R₅, and R₆ are independently hydrogen, halogen, hydroxyl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
R₇ and R₈ are independently hydrogen, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; or R₇ and R₈ are taken together with the N to which they are attached to form a ring system.

2. The compound of claim 1, wherein the compound is of formula 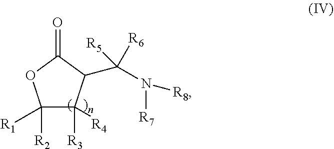
or a pharmaceutically acceptable salt thereof,
wherein
n is 1, 2, 3, or 4;
R₁, R₂, R₃, R₄, R₅, and R₆ are independently hydrogen, halogen, hydroxyl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
R₇ and R₈ are independently hydrogen, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyi, arylalkenyl, or arylalkynyl, each of which is optionally substituted: or R₇ and R₈ are taken together with the N to which they are attached to form a ring system.

3. The compound of claim 1, wherein the compound is of formula 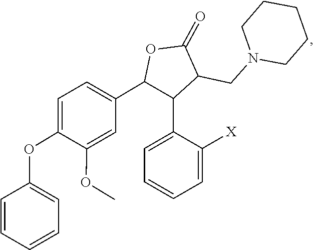
or a pharmaceutically acceptable salt thereof, wherein is a halo.

4. The compound of claim 1, wherein the compound is of formula 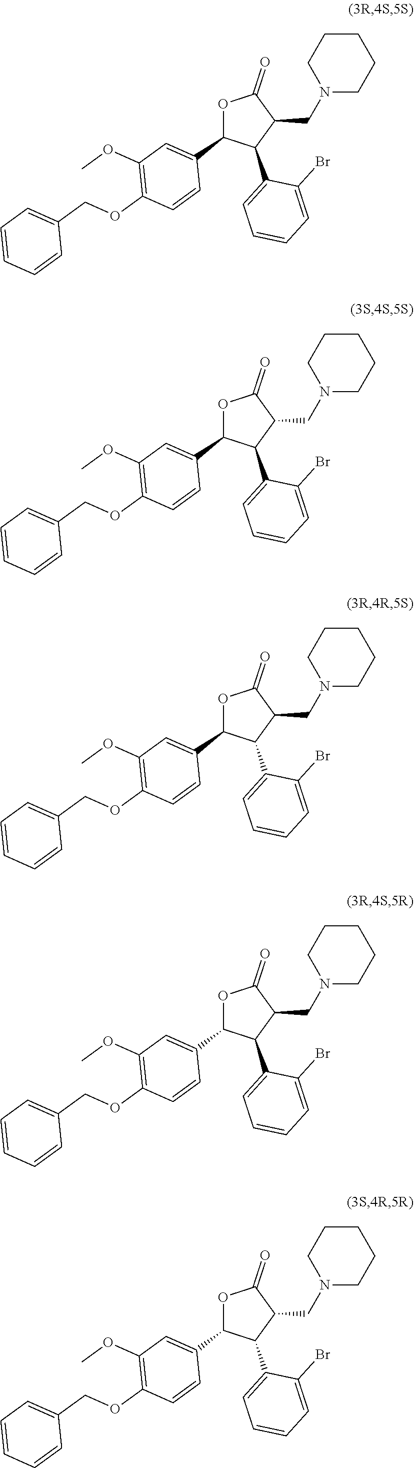 [SMILES: COc1cc([C@H]2OC(=O)[C@@H](CN3CCCCC3)[C@H]2c2ccccc2Br)ccc1OCc1ccccc1 ; COc1cc([C@H]2OC(=O)[C@H](CN3CCCCC3)[C@H]2c2ccccc2Br)ccc1OCc1ccccc1 ; COc1cc([C@H]2OC(=O)[C@@H](CN3CCCCC3)[C@@H]2c2ccccc2Br)ccc1OCc1ccccc1 ; COc1cc([C@@H]2OC(=O)[C@@H](CN3CCCCC3)[C@H]2c2ccccc2Br)ccc1OCc1ccccc1 ; COc1cc([C@@H]2OC(=O)[C@H](CN3CCCCC3)[C@@H]2c2ccccc2Br)ccc1OCc1ccccc1] 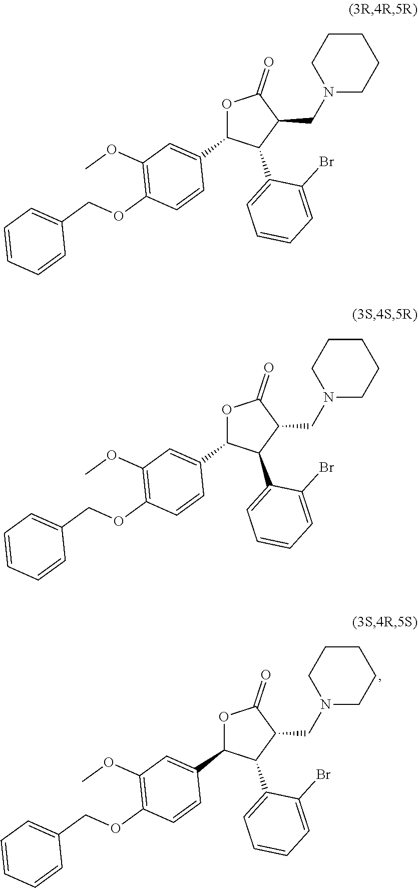 [SMILES: COc1cc([C@@H]2OC(=O)[C@@H](CN3CCCCC3)[C@@H]2c2ccccc2Br)ccc1OCc1ccccc1 ; COc1cc([C@@H]2OC(=O)[C@H](CN3CCCCC3)[C@H]2c2ccccc2Br)ccc1OCc1ccccc1 ; COc1cc([C@H]2OC(=O)[C@H](CN3CCCCC3)[C@@H]2c2ccccc2Br)ccc1OCc1ccccc1]
or a pharmaceutically acceptable salt thereof.

5. A compound of formula (I): 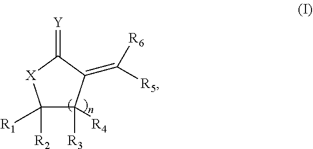
wherein
X is O, NH, NR, or S;
Y is O, NH, NR, or S;
n is 1, 2, 3, or 4; and
R₁, R₂, R₃, R₄, R₅, and R₆ are independently hydrogen, halogen, hydroxyl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted.

6. The compound of claim 5, wherein the compound is of formula 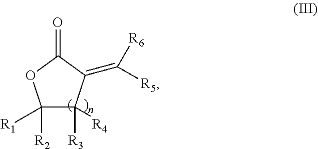
or a pharmaceutically acceptable salt thereof,
wherein
n is 1, 2, 3, or 4; and
R₁, R₂, R₃, R₄, R₅, and R₆ are independently hydrogen, halogen, hydroxyl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl.

7. A pharmaceutical composition comprising one or more compounds of claim 1, together with one or more pharmaceutically acceptable diluents, excipients or carriers.

8. The pharmaceutical composition of claim 7, which is nanoparticulate.

9. The pharmaceutical composition of claim 7, further comprising one or more other antibiotics.

10. A method for treating a patient with an infection comprising administering a therapeutically effective amount of one or more compounds of claim 1, or a pharmaceutically acceptable salt thereof.

11. The method of claim 10, wherein said infection is caused by Clostridioides difficile.

12. (canceled)

13. A pharmaceutical composition comprising one or more compounds of claim 5, together with one or more pharmaceutically acceptable diluents, excipients or carriers.

14. The pharmaceutical composition of claim 7, which is nanoparticulate.

15. The pharmaceutical composition of claim 7, further comprising one or more other antibiotics.

16. A method for treating a patient with an infection comprising administering a therapeutically effective amount of one or more compounds of claim 5, or a pharmaceutically acceptable salt thereof.

17. The method of claim 16, wherein said infection is caused by Clostridioides difficile.
